(19) United States
(12) Patent Application Publication
(10) Pub. No.: US 2023/0390291 A1
(43) Pub. Date: Dec. 7, 2023

(54) TREATMENT OF SKIN DISORDERS WITH COMPOSITIONS COMPRISING AN EGFR INHIBITOR

(71) Applicant: Sol-Gel Technologies Ltd., Ness Ziona (IL)

(72) Inventors: Moshe ARKIN, Kfar Shmaryahu (IL); Marcel ZIGHELBOIM, Kiryat Motzkin (IL); Ori NOV, Tarum (IL); Ofer TOLEDANO, Kfar Saba (IL); Karine NEIMANN, Ness Ziona (IL)

(73) Assignee: Sol-Gel Technologies Ltd., Ness Ziona (IL)

(21) Appl. No.: 18/453,355

(22) Filed: Aug. 22, 2023

Related U.S. Application Data
(63) Continuation-in-part of application No. 17/352,322, filed on Jun. 20, 2021.
Application No. 17/352,322 is a continuation-in-part of application No. 16/737,503, filed on Jan. 8, 2020, now Pat. No. 11,633,399.
Application No. 16/737,503 is a continuation-in-part of application No. PCT/IL19/51410, filed on Dec. 25, 2019.
(60) Provisional application No. 62/877,957, filed on Jul. 24, 2019.
(60) Provisional application No. 62/877,990, filed on Jul. 24, 2019.
(60) Provisional application No. 62/784,738, filed on Dec. 25, 2018.

(51) Int. Cl. A61K 31/517; A61P 35/00; A61K 9/00

(52) U.S. Cl. CPC A61K 31/517; A61P 35/00; A61K 9/0014

(57) ABSTRACT

Provided herein compositions and methods of treatment of skin or mucosal disorders by administration of compositions comprising at least one EGFR inhibitor, such as topical compositions comprising erlotinib. The compositions of this invention are useful for the treatment, prevention or amelioration of skin or mucosal disorders like psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa.

### CROSS REFERENCE TO RELATED APPLICATIONS

This Application is a Continuation-in-Part Application of U.S. application Ser. No. 17/352,322, filed Jun. 20, 2021, which is a Continuation-in-Part Application of U.S. application Ser. No. 16/737,503, filed Jan. 8, 2020, which is a Continuation-in-Part of PCT International Patent Application No. PCT/IL2019/051410, filed on Dec. 25, 2019, which claims the benefit of U.S. Provisional Application Ser. No. 62/877,957, filed on Jul. 24, 2019, U.S. Provisional Application Ser. No. 62/877,990, filed on Jul. 24, 2019, and U.S. Provisional Application Ser. No. 62/784,738, filed Dec. 25, 2018, which are all incorporated in their entirety herein by reference.

### FIELD OF THE INVENTION

This invention, in some embodiments thereof, relates to compositions and methods of treatment of skin or mucosal disorders by administration of compositions comprising at least one EGFR inhibitor, such as topical compositions comprising erlotinib. The compositions of this invention are useful for the treatment, prevention or amelioration of skin or mucosal disorders like psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa.

The subject invention is directed to treatment of non-melanoma skin cancer using topical EGFR inhibitor.

### BACKGROUND

Epidermal Growth Factor Receptor (EGFR) inhibitor drugs like erlotinib, gefitinib, osimertinib and brigatinib target the EGFR (a known oncogene) and are used for the systemic treatment of some forms of cancer (lung, colon).

There is no US-marketed EGFR inhibitor drug for topical use or for injection. The EGFR inhibitor erlotinib is sold as oral tablets (Tarceva). Similarly, gefitinib (Iressa), osimertinib (Tigresso) and brigatinib (Alunbrig) are sold as oral tablets.

Erlotinib, having the following structure:

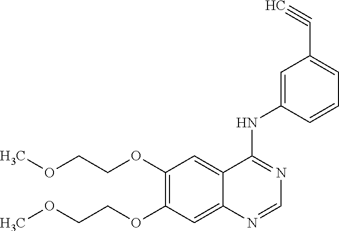 [SMILES: C#Cc1cccc(Nc2ncnc3cc(OCCOC)c(OCCOC)cc23)c1]

chemically N-(3-ethynylphenyl)-6,7-bis(2-methoxyethoxy)-4-quinazolinamine, is indicated for the treatment of patients with locally advanced or metastatic non-small cell lung cancer after failure of at east one prior chemotherapy regimen, and in combination with gemcitabine is indicated for the first-line treatment of patients with locally advanced, unresectable or metastatic pancreatic cancer.

Erlotinib is administered as its hydrochloride salt and is currently marketed as TARCEVA® (erlotinib) tablets. Erlotinib hydrochloride has the molecular formula C₂₂H₂₃N₃O₄·HCl and a molecular weight of 429.90. It is very slightly soluble in water, slightly soluble in methanol and practically insoluble in acetonitrile, acetone, ethyl acetate and hexane.

WO 2009/091889 discloses methods and compositions for the treatment of skin disorders (e.g., genetic skin disorders) such as Darier's disease. The treatment for skin genetic disorder can also be applied to patients further diagnosed to have cancer. In such cases, a combination therapy of EGFR inhibitor(s) (for the treatment of the genetic skin disorder), and an anti-cancer agent (for the treatment of cancer) is considered. (Page 15 and 23 of WO '889).

There is an unmet need for methods of topical or injectable treatment of skin or mucosal disorders like psoriasis, palmoplantar psoriasis, acquired palmoplantar keratosis, eczema, ichthyosis vulgaris, non-melanoma skin cancer, basal cell carcinoma (BCC), squamous cell skin cancer (SCC), Merkel cell carcinoma, Bowen's disease, classic kaposi sarcoma, hereditary leiomyomatosis (Reed's syndrome), Ankyloblepharon-ectodermal defects-cleft lip/palate (AEC) syndrome, dermatofibrosarcoma protuberans, Granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma or sebaceous carcinomaactinic keratosis, a keratinization skin disorder, Darier's disease, Hailey-Hailey disease, palmoplantar keratoderma a keratinization mucosal disorder, pachyonychia congenita, hidradenitis suppurativa, Gorlin syndrome, prurigo nodularis and prurigo pigmentosa.

### SUMMARY OF THE INVENTION

In one embodiment, this invention is directed to a method of treating, preventing or alleviating non-melanoma skin cancer, wherein the method comprises topical administration to a subject in need thereof, a therapeutically effective amount of a topical composition comprising erlotinib or a pharmaceutically acceptable salt thereof in a concentration of about 0.1% w/w to about 20% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In one further embodiment, said non-melanoma skin cancer is skin cancer basal cell carcinoma (BCC), squamous cell skin cancer (SCC), actinic keratosis, Gorlin syndrome, Merkel cell carcinoma, Bowen's disease, classic Kaposi sarcoma, hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, or sebaceous carcinoma.

In one embodiment, provided herein a topical composition comprising erlotinib or a pharmaceutically acceptable salt thereof in a concentration of about 3.5%-7.5% w/w, and a pharmaceutically acceptable carrier, wherein administration of said composition does not induce or induces reduced cutaneous side-effects as compared with systemic administration of the same amount of erlotinib or pharmaceutically acceptable salt thereof and wherein said administration does not induce cutaneous toxicity or induces reduced cutaneous toxicity of erlotinib or pharmaceutically acceptable salt thereof. In other embodiments, the topical composition comprises water in a concentration of about 0% w/w to about 4% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 3.5% w/w to about 7% w/w. In other embodiments, the topical composition comprises water in a concentration of about 0% w/w to about 4% w/w. In another embodiment, the topical composition has a systemic absorption of less than 100 ng/mL of erlotinib or less than 10 ng/mL of erlotinib metabolite.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1E presents a significant decrease in proliferation index (Ki-67) in Erlotinib-treated xenotransplants compared to vehicle-treated xenotransplants. FIGS. 1A and 1B present vehicle-treated xenotransplants after 14 days of treatment. FIG. 1C presents Erlotinib-treated xenotransplants after 14 days of treatment, in complete recovery. FIG. 1D presents Erlotinib-treated xenotransplants after 14 days of treatment. FIG. 1E presents a proliferation index (Ki-67) in Erlotinib-treated xenotransplants (at different concentrations of 0.75%, 3.5% and 5% w/w) compared to vehicle-treated xenotransplants. The data are presented as the mean ±standard deviation. The scale bars are 50 μm. The study parameters of each group were compared using the Kruskal-Wallis test, followed by the Mann-Whitney U test. Statistical significance was set at p<0.05.

### DETAILED DESCRIPTION OF THE INVENTION

Treatment with EGFR inhibitors in general and erlotinib in particular is known to induce cutaneous conditions like acneiform rash, papulopustular rash, abnormal scalp hair growth, abnormal facial hair growth, abnormal hair growth, abnormal eyelash growth, paronychia with or without pyogenic granulomas and telangiectasia.

This is probably one of the reasons that no topical EGFR inhibitor product is developed or marketed so far.

It occurred to the present inventors that EGFR inhibitors, being tyrosine kinase inhibitors and also essential regulators of multiple epidermal functions have the potential to prevent, cure or alleviate a number of skin or mucosal disorders in which tyrosine kinase inhibition or epidermal function regulation play a causal mechanistic role.

Surprisingly, according to the present invention, even by increasing the EGFR inhibitors (such as erlotinib) concentration up to 5 wt % or even up to 10 wt %, the cutaneous side effects were not increased and no or very low systemic absorption was observed. This is of particular importance because erlotinib is known to have significant side effects that may also occur in topical therapy.

The EGFR inhibitor cutaneous side-effects reported in the medical literature are the result of oral (systemic) treatment with EGFR inhibitors. The compositions and methods of treatment of this invention use non- oral administration, thus avoiding systemic effects, and are therefore expected to present an advantageous cutaneous side-effects profile as compared to the EGFR inhibitor oral products.

Some of the skin or mucosal disorders contemplated for treatment with the methods of this invention are psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa (see below).

### Psoriasis

Psoriasis is characterized i.e. by epidermal hyperproliferation. Protein tyrosine kinases (PTKs) regulate cell proliferation, differentiation and immune processes. Uncontrolled signaling from receptor and intracellular tyrosine kinases can lead to numerous proliferative diseases, i.a. psoriasis (Ben-Bassat H et al Curr. Pharm Des. 2000 Jun;6(9):933-42).

### Palmoplantar Psoriasis

Palmoplantar psoriasis is a variant of psoriasis that characteristically affects the skin of the palms and soles. It features hyperkeratotic, pustular, or mixed morphologies. The condition is chronic in nature and produces significant functional disability (see Miceli A, Schmieder G J. Palmoplantar Psoriasis. [Updated 2019 Jun 3]. StatPearls [Internet]. Treasure Island (FL): StatPearls Publishing; 2019 Jan). https://www.ncbi.nlm.nih.gov/books/NBK448142/

### Hidradenitis Suppurativa (HS)

Hidradenitis suppurativa (HS), also known as acne inversa, is a long-term chronic skin disease whose present treatment options are often unsatisfactory. HS has a profound effect on patient's quality of life (QoL). Alavi A. et al., reviewed QoL aspects of this disease in an article titled “Quality-of-Life Impairment in Patients with Hidradenitis Suppurativa” (Am J Clin Dermatol, 2014, vol. 15.No. 6).

The clinical picture of HS includes solitary nodules, diffuse, painful abscesses, malodorous drainage, sinus tract formation and scarring.

The exact cause of hidradenitis suppurativa is still unclear, but it is believed that the underlying mechanism involves dysfunction of the apocrine sweat glands or hair follicles.

### Pachyonychia Congenita

Pachyonychia Congenita (PC) is an ultra-rare genetic autosomal dominant skin disorder. PC is caused by a mutation in one of five keratin genes KRT6A, KRT6B, KRT6C, KRT16 or KRT17. Keratin genes are responsible for production of keratins, which are tough, fibrous proteins that form filaments to support skin cells and give them shape and strength. Keratin filaments help cells handle pressure and stretching. With PC, the filaments do not form properly, causing extreme cell fragility. PC is a congenital autosomal dominant syndrome primarily affecting males, mainly characterized by increased thickness of the nails, hyperkeratosis involving the palms, soles, knees, and elbows, with popular tiny cutaneous horns, leukoplakia of the mucous membranes (leukokeratosis of the oral mucosa), and usually excessive sweating of the hands and feet; associated with development of bullae on palms and soles after trauma, also characterized by cysts of various types (including steatocystoma and pilosebaceous cysts), characterized by follicular hyperkeratosis (FHK or bumps around hairs at friction sites such as waist, hips, knees, elbows). Most common in children and lessens after teenage years.

In some embodiments, the PC is associated with nail dystrophy. In other embodiments the PC is associated with a keratinization skin disorder.

### Keratinization Skin Disorders

This class of skin disorders includes hyperkeratinization disorder, Darier's disease, Hailey-Hailey disease, erythrodermic autosomal recessive lamellar ichthyosis, nonerythrodermic autosomal recessive lamellar ichthyosis, autosomal dominant lamellar ichthyosis, bullous congenital ichthyosiform erythroderma, palmoplantar keratoderma, erythrokeratodermia variabilis, verrucous epidermal nevi, pityriasis rubra pilaris, Netherton syndrome, idiopathic vulgaris, ichthyosis vulgaris, monilethrix, keratosis piliaris, bullous ichthyosiform erythroderma, nonbullous congenital ichthyosis, Sjogren-Larsson syndrome, erythrokeratodermica variabilis, hyperkeratosis lenticularis perstans, eythrokeratodermia figurate variabilis, mutilating keratoderma of Vohwinkel, Harlequin ichthyosis and Tay's syndrome.

A new terminology for the keratinization skin disorders has been recently introduced (see Akiyama M. et al., J Dermatol Sci. 2018 May;90(2):105-111, “Autoinflammatory keratinization diseases: An emerging concept encompassing various inflammatory keratinization disorders of the skin”).

In one embodiment the keratinization skin disorder includes Pachyonychia Congenita (PC).

### Keratinization Mucosal Disorders

This class of mucosal (oral, vaginal, anal) disorders includes Lichen Planus, Leukoplakia and Lichen sclerosus.

### Prurigo Nodularis

Prurigo nodularis is a skin disease characterised by pruritic (itchy) nodules which usually appear on the arms or legs. Patients often present with multiple excoriated lesions caused by scratching. Prurigo nodularis is very hard to treat, but current therapies include steroids, vitamins, cryosurgery, thalidomide and UVB light.

### Prurigo Pigmentosa

Prurigo pigmentosa is a rare skin condition of unknown cause, characterized by the sudden onset of erythematous papules that leave a reticulated hyperpigmentation when they heal.

Non-Melanoma Skin Cancer (NMSC)

Skin cancers include three main types—basal-cell skin cancer (BCC), squamous cell skin cancer (SCC) and melanoma.

The first two types together (BCC and SCC) are known as non-melanoma skin cancers (NMSC).

Cetuximab (Erbitux), an EGFR inhibitor, has been investigated for oral treatment of NMSC (Wollina U., Expert Opinion on Biological Therapy, Vol. 14, 2014—Issue 2).

Uncontrolled signaling from receptor and intracellular tyrosine kinases can lead to numerous proliferative diseases, i.e. cancer (Ben-Bassat H et al Curr. Pharm Des. 2000 Jun;6(9):933-42).

Nonmelanoma skin cancer can refer to any cancer that forms in the basal layer (BCC), squamous layer (SCC) or Merkel cells of the skin. Melanoma is a relatively common cancer that begins in the melanocytes, which are the pigment-producing cells located on the top layer of the skin. Diseases related to non-melanoma skin cancer are diseases that for an internal factor such as genetics, hereditary or even external factor such as the sun, repeated trauma they can be transformed into BCC or SCC.

The following disease are non-melanoma skin cancer or related to non-melanoma skin cancer:

### Actinic Keratosis (AK)

Actinic keratosis with exposure to the sun can transform into BCC and/or SCC. Dysregulation of the EGFR signaling results in cellular hyperproliferation and defects in differentiation (Joseph S R et al., “Dysregulation of epidermal growth factor receptor in actinic keratosis and squamous cell carcinoma”, Curr Probl. Dermatol. 2015; 46:20-7)

### Gorlin Syndrome (NBCCS)

NBCCS (Nevoid Basal-Cell Carcinoma Syndrome) is a predisposition for BCC caused by a genetic mutation. Oral treatment of NMSC (which includes BCC) with cetuximab (an EGFR inhibitor) has been investigated, but the topical treatment of Gorlin syndrome with topical EGFR inhibitors was never attempted.

### Merkel Cell Carcinoma

Merkel cell carcinoma is a rare type of skin cancer that usually appears as a flesh-colored or bluish-red nodule, often on human face, head or neck. Sun exposure and/or a weak immune system can increase the risk of developing Merkel cell carcinoma. Merkel cell carcinoma usually appears as a single painless lump on sun-exposed skin.

### Keratocystic Odontogenic Tumor

KOT (keratocystic odontogenic tumor) is a one of three cardinal features of Gorlin Syndrome. The other two three cardinal features of Gorlin Syndrome are Multiple BCC and Palmar Plantar pits.

### Bowen's Disease

Bowens disease is a very early form of skin cancer. The main sign is a red, scly patch on the skin. It affects the squamous cells, which are in the outer sayer of skin, and is sometimes referred to as squamous cell carcinoma in situ. The patch is usually slow growing, but there's a chance it could turn into a more serious type of skin cancer if left untreated.

### Classic Kaposi Sarcoma

Kaposi sarcoma (KS) is a rare, treatable cancer that can affect people with weakened. immune systerns. The classic Kaposi sarcoma type, usually diagnosed in older men of Mediterranean, Middle Eastern and Eastern European descent. People who have classic Kaposi sarcoma typically have slow-growing skin lesions that grow in size and number. Lesions might also spread to internal organs.

### Hereditary Leiomyomatosis (Reed's Syndrome)

Hereditary leiomyomatosis and renal cell cancer (HLRCC) is a disorder in which affected individuals tend to develop benign tumors containing smooth muscle tissue (leiomyomas) in the skin and, in females, the uterus . This condition also increases the risk of kidney cancer.

### Ankyloblepharon-Ectodermal Defects-Cleft Lip/Palate (AEC) Syndrome

Ankyloblepharon-ectodermal defects-cleft lip/palate syndrome (AEC syndrome) is a form of ectodermal dysplasia, a group of conditions characterized by abnormal development of ectodermal tissues including the skin, hair, nails, teeth, and sweat glands. The most common feature is missing patches of skin (erosions) on the scalp, neck, hands, and feet. The skin erosions can lead to infection, scarring, and hair loss. Other features include changes in skin coloring; misshapen or absent fingernails and toenails; malformed or missing teeth; increased sensitivity to heat; hearing loss; cleft lip and/or palate; and other facial abnormalities. This condition is caused by genetic changes in the TP63 gene and is inherited in an autosomal dominant fashion. Rapp-Hodgkin syndrome was classified as a separate disorder until it was discovered that it results from genetic changes in the same part of the TP63 gene. Rapp-Hodgkin syndrome and AEC syndrome are considered to be part of the same disease spectrum.

### Dermatofibrosarcoma Protuberans

Dermatofibrosarcoma protuberans (DFSP) is a rare type of skm cancel. It starts in connective tissue cells in the middle layer of the skin (dermis). The tumor typically presents as a slowly growing, firm plaque on the trunk of young adults. The cause of dermatofibrosarcoma protuberans is not clearly understood.

### Granular Cell Tumors on Skin

Granular cell tumors are rare, generally benign, soft tissue neoplasms believed to originate from Schwann cells (cells that provide myelin insulation to nerves). They can occur in the skin, tongue, breast, gastrointestinal tract, and respiratory tract.

### Disseminated Superficial Actinic Porokeratosis (DSAP)

Disseminated superficial actinic porokeratosis, or DSAP, is an inherited keratinisation disorder that causes discrete dry patches on the arms and legs.

The development of squamous cell carcinoma (SCC) within a DSAP lesion is the main concern. This is uncommon (<10% of individuals with DSAP develop SCC). However, many patients with DSAP have had significant exposure to the sun and may also have actinic keratoses and other forms of skin cancer (particularly basal cell carcinoma). SCC presents as a solitary tender enlarging scaly or ulcerated plaque or nodule.

### Sezary Syndrome

Sezary syndrome is an erythrodermic cutaneous T-cell lymphoma with the leukemic component. Erythroderma is an intense, widespread, pruritic, exfoliative rash representing new lesions or progression of the prior patches or plaques.

### Epidermolysis Bullosa-Associated Squamous Cell Carcinoma

Epidermolysis bullosa (EB) is a heterogeneous group of inherited skin disorders determined by mutations in genes encoding for structural components of the cutaneous basement membrane zone.

Almost all subjects affected with the severe recessive dystrophic EB (RDEB) subtype

suffer from early and extremely aggressive SCCs (RDEB-SCC), which represent the first cause of death in these patients. Usually, the tumors are localized at sites of chronic, hard-to-heal wounds and scarring, they are frequently multiple, and, specific to recessive DEB, highly aggressive representing the first cause of death in this EB subtype.

In EB patients, skin fragility leads to many possible complications and comorbidities. One of the most feared complications is the development of cutaneous squamous cell carcinomas (SCCs) that particularly in the dystrophic recessive EB subtype can be extremely aggressive and often metastatic. SCCs in EB patients generally arise more often in the extremities, where chronic blisters and scars are generally located. SCCs represent a big therapeutic challenge in the EB population.

### Angiosarcoma of the Skin

This type of cancer can occur any place in the body. But it most often occurs in the skin on the head and neck.

### Cutaneous B-cell Lymphoma

Cutaneous B-cell lymphoma is a rare type of cancer that begins in the white blood cells. This cancer attacks the skin. Cutaneous B-cell lymphoma symptoms include a firm bump under the skin. The bump might be the same color as your skin. Or it might be a darker color or look pink or purple.

### Cutaneous T-cell Lymphoma

Cutaneous T-cell lymphoma (CTCL) is a rare type of cancer that begins in white blood

cells called T cells (T lymphocytes). n cutaneous T-cell lymphoma, the T cells develop abnormalities that make them attack the skin. Cutaneous T-cell lymphoma can cause rash-like skin redness, slightly raised or scaly round patches on the skin, and, sometimes, skin tumors. The most common type is mycosis fungoides.

### Sebaceous Carcinoma

is a rare type of cancer that begins in an oil gland in the skin. Sebaceous carcinoma most often affects the eyelids.Sebaceous carcinoma may begin as a painless lump or thickening of skin on the eyelid. On other parts of the body it might cause a bump on the skin that may bleed or have a scab.

In some embodiments, the EGFR inhibitor in this invention is selected from gefitinib, erlotinib, lapatinib, cetuximab, panitumumab, vandetanib, necitumumab, osimertinib and combinations thereof.

According to some embodiments, there are provided novel methods of treatment of above enumerated skin or mucosal disorders by topical administration of at least one EGFR inhibitor.

According to some embodiments, there is provided a method of treating, preventing or alleviating non-melanoma skin cancer selected from the group consisting of basal cell carcinoma (BCC), squamous cell skin cancer (SCC), actinic keratosis, Gorlin syndrome, Merkel cell carcinoma, Bowen's disease, classic Kaposi sarcoma, Hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma, comprises topical administration to a subject in need thereof a therapeutically effective amount of a topical composition comprising erlotinib or a pharmaceutically acceptable salt thereof in a concentration of about 0.1% w/w to about 20% w/w. In other embodiments, the composition comprises erlotinib or a pharmaceutically acceptable salt thereof in a concentration of about 3.5% w/w to about 7.5% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

According to some embodiments, there is provided a method of treating, preventing or alleviating non-melanoma skin cancer selected from the group consisting of basal cell carcinoma (BCC), squamous cell skin cancer (SCC), actinic keratosis, Gorlin syndrome, Merkel cell carcinoma, Bowen's disease, classic Kaposi sarcoma, Hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma, comprises topical administration to a subject in need thereof a therapeutically effective amount of a topical composition comprising erlotinib or a pharmaceutically acceptable salt thereof in a concentration of about 0.1% w/w to about 20% w/w. wherein said subject does not suffer from a skin keratinization disorder that requires concomitant treatment of the skin keratinization disorder. In another embodiment, the skin keratinization disorder is genetic skin keratinization disorder. In other embodiments, the composition comprises erlotinib or a pharmaceutically acceptable salt thereof in a concentration of about 3.5% w/w to about 7.5% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

According to some embodiments, there is provided a method of treating, preventing or alleviating non-melanoma skin cancer selected from the group consisting of basal cell carcinoma (BCC), squamous cell skin cancer (SCC), actinic keratosis, Gorlin syndrome, Merkel cell carcinoma, Bowen's disease, classic Kaposi sarcoma, Hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma, comprises topical administration to a subject in need thereof a therapeutically effective amount of a topical composition comprising erlotinib or a pharmaceutically acceptable salt thereof in a concentration of about 0.1% w/w to about 20% w/w, and wherein the method decreases proliferation index (Ki67). In other embodiments, the subject does not suffer from a skin keratinization disorder that requires concomitant treatment of the skin keratinization disorder. In another embodiment, the skin keratinization disorder is genetic skin keratinization disorder.

In some embodiments, the method provided herein reduces proliferation index (Ki-67).

Ki67 is a well-known proliferation marker for the evaluation of cell proliferation. Numerous studies have indicated that Ki67 index independently predicts cancer progression. Moreover, because Ki67 is highly expressed in malignant cells but almost could not be detected in normal cells, it has become a promising target for cancer therapy.

In some embodiments, provided herein a method for reducing the proliferation index (Ki67) in a subject, comprising administering a topical composition comprising erlotinib or a pharmaceutically acceptable salt thereof in a concentration of about 3.5% to about 7.5% w/w, and a pharmaceutically acceptable carrier. In other embodiments, the composition comprising erlotinib or a pharmaceutically acceptable salt thereof in a concentration of about 3.5% to about 7.5% w/w, reduces the proliferation index (Ki67) by more than 10% , 20%, 30% compared to a composition comprising less than 1% erlotinib or a pharmaceutically acceptable salt thereof. In other embodiments, the composition comprising erlotinib or a pharmaceutically acceptable salt thereof in a concentration of about 3.5% to about 7.5% w/w, reduces the proliferation index (Ki67) by more than 10%, 20%, 30% compared to a composition comprising 0.75% w/w erlotinib or a pharmaceutically acceptable salt thereof.

According to some embodiments, there is provided a method of reducing proliferation index (Ki67) comprises topical administration to a subject in need thereof a therapeutically effective amount of a topical composition comprising erlotinib or a pharmaceutically acceptable salt thereof in a concentration of about 3.5 w/w to about 7.5% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

According to some embodiments, there is provided a method of reducing proliferation index (Ki67) comprises topical administration to a subject in need thereof a therapeutically effective amount of a topical composition comprising erlotinib or a pharmaceutically acceptable salt thereof in a concentration of about 3.5 w/w to about 7.5% w/. In another embodiment, the skin keratinization disorder is genetic skin keratinization disorder. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

According to some embodiments, there is provided a method of reducing proliferation index (Ki67) comprises topical administration to a subject in need thereof a therapeutically effective amount of a topical composition comprising erlotinib or a pharmaceutically acceptable salt thereof in a concentration of about 3.5 w/w to about 7.5% w/w, wherein said subject does not suffer from a skin keratinization disorder that requires concomitant treatment of the skin keratinization disorder. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

According to some embodiments, there are provided novel methods of treatment of the above non-melanoma skin cancer enumerated disorders to a subject in need thereof by topical administration of a therapeutically effective amount of a topical composition comprising erlotinib or a pharmaceutically acceptable salt thereof in a concentration of about 0.1% w/w to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from about 3.5% to about 7.5% w/w, from 4% to about 6% w/w, from about 0.5% w/w to about 6% w/w, from about 0.75% w/w to about 5.5% w/w, about 3% w/w to about 6% w/w, about 5% w/w to about 10% w/w, or about 10% w/w to about 20% w/w. In another embodiment, said subject does not suffer from a skin keratinization disorder that requires concomitant treatment of the skin keratinization disorder. In another embodiment, the skin keratinization disorder is genetic skin keratinization disorder.

According to some embodiments, there are provided novel methods of treatment of above non-melanoma skin cancer enumerated disorders by topical administration of a therapeutically effective amount of a topical composition comprising erlotinib or a pharmaceutically acceptable salt thereof in a concentration from about 0.1% w/w to about 20% w/w from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from about 3.5% to about 7.5% w/w, from 4% to about 6% w/w, from about 0.5% w/w to about 6% w/w, from about 0.75% w/w to about 5.5% w/w, about 3% w/w to about 6% w/w, from about 5% to about 10% w/w or from about 10% to about 20% w/w, water in a concentration from about 0% w/w to about 4% w/w, from about 0% to about 0.1% w/w, from about 0% to about 2.5% w/w, from about 0.1% to about 1% w/w, from about 0.1% to about 2%, from about 2% to about 3%, from about 3% to about 4% w/w, from 0% to about 3% w/w, from about 1.5% to about 3% w/w and a pharmaceutically acceptable carrier. Each possibility represents a separate embodiment of this invention. In another embodiment, the administration has a systemic absorption of less than 100 ng/mL of erlotinib or less than 10 ng/mL of erlotinib metabolite in the plasma. In another embodiment, the administration has a systemic absorption of less than 1% or less than 0.5% by weight of erlotinib or pharmaceutically acceptable salt thereof.

According to some embodiments, there are provided novel methods of treatment of above non-melanoma skin cancer enumerated disorders by topical administration of a therapeutically effective amount of a topical composition comprising erlotinib or a pharmaceutically acceptable salt thereof in a concentration from about 3.5% to about 7.5% w/w. In other embodiments, at concentration of 3.5%, 4%, 4.5%, 5%, 5.5%, 6%, 6.5%, 7% or 7.5% w/w. In another embodiment, the administration has a systemic absorption of less than 100 ng/mL of erlotinib or less than 10 ng/mL of erlotinib metabolite in the plasma. In another embodiment, the administration has a systemic absorption of less than 1% or less than 0.5% by weight of erlotinib or pharmaceutically acceptable salt thereof.

According to some embodiments, there are provided novel methods of treatment of above non-melanoma skin cancer enumerated disorders by topical administration of a therapeutically effective amount of a topical composition comprising erlotinib or a pharmaceutically acceptable salt thereof in a concentration from about 5% w/w. In another embodiment, the administration has a systemic absorption of less than 100 ng/mL of erlotinib or less than 10 ng/mL of erlotinib metabolite in the plasma. In another embodiment, the administration has a systemic absorption of less than 1% or less than 0.5% by weight of erlotinib or pharmaceutically acceptable salt thereof.

According to some embodiments, there are provided novel methods of treatment of above non-melanoma skin cancer enumerated disorders by topical administration of a therapeutically effective amount of a topical composition comprising erlotinib or a pharmaceutically acceptable salt thereof in a concentration from about 0.1% w/w to about 20% w/w from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from about 3.5% to about 7.5% w/w, from 4% to about 6% w/w, from about 0.5% w/w to about 6% w/w, from about 0.75% w/w to about 5.5% w/w, about 3% w/w to about 6% w/w, from about 5% to about 10% w/w or from about 10% to about 20% w/w, water in a concentration from about 0% w/w to about 4% w/w, from about 0% to about 0.1% w/w, from about 0% to about 2.5% w/w, from about 0.1% to about 1% w/w, from about 0.1% to about 2%, from about 2% to about 3%, from about 3% to about 4% w/w, from 0% to about 3% w/w, from about 1.5% to about 3% w/w and a pharmaceutically acceptable carrier, wherein the method reduces the proliferation index (Ki-67). In another embodiment, the administration has a systemic absorption of less than 100 ng/mL of erlotinib or less than 10 ng/mL of erlotinib metabolite in the plasma. In another embodiment, the administration has a systemic absorption of less than 1% or less than 0.5% by weight of erlotinib or pharmaceutically acceptable salt thereof.

In some embodiments there is provided a method of treatment of a skin or mucosal disorder in which epidermal function regulation or tyrosine kinase inhibition play a causal mechanistic role, by topical administration of a therapeutically effective amount of at least one EGFR inhibitor.

According to some embodiments, there is provided a topical composition for treatment, prevention or alleviation of a skin or mucosal disorder selected from the group consisting of psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa, comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w. In another embodiment, the keratinization skin disorder is hyperkeratinization disorder. Each possibility represents a separate embodiment of this invention. In some embodiments, the topical composition further comprises at least one penetration enhancer. In one embodiment, the penetration enhancer is in a concentration of between 10% w/w to about 98% w/w of said composition. In some embodiments, the topical composition further comprises at least one keratolytic agent. In one embodiment, the keratolytic agent is in a concentration of between 0.1% w/w to about 40% w/w of said composition.

In one other embodiment, the at least one penetration enhancer is selected from: DMSO (dimethyl sulfoxide), ethanol, isopropyl alcohol, dimethyl isosorbide, isopropyl myristate, oleic acid, a polyethylene glycol, hexylene glycol, glycofurol and combinations thereof. In one another embodiment, the at least one penetration enhancer has dual functionality and may act also as solvent. Each possibility represents a separate embodiment of this invention.

In some embodiments, the composition of this invention and method of use thereof comprises a keratolytic agent.

As used herein a “keratolytic agent” refers to a compound which loosens and removes the stratum corneum of the skin or alters the structure of the keratin layers of skin. Suitable keratolytic agents include alpha-hydroxy acids. Non-limiting examples of alpha-hydroxy acids include lactic acid and glycolic acid, malic acid, citric acid and tartaric acid. Alfa hydroxyl acids are keratolytic, and they are also capable of trapping moisture in the skin and initiating the formation of collagen. Another group of keratolytic agents, suitable for inclusion in the therapeutic composition according to the present invention is beta-hydroxy acids, such as Salicylic acid (o-hydroxybenzoic acid). Beta hydroxyl acids are keratolytic, and they also have anti-inflammatory and antibacterial properties. Short chain carboxylic acids (carboxylic acids having up to 6 carbon atoms in their skeleton) are also suitable for inclusion in the therapeutic composition as keratolytic agents. Examples of short chain carboxylic acid include, but are not limited to formic acid, acetic acid, propionic acid, butyric acid (Butanoic acid), valeric acid (pentanoic acid) and caproic acid (hexanoic acid). Also suitable under the definition of short chain carboxylic acid are unsaturated short chain carboxylic acids, i.e., short chain carboxylic acids, having one or more double bonds in their carbon skeleton; and halogenated short chain carboxylic acids, such as fluoroethanoic acid (CH₂FCO₂H), chloroethanoic acid (CH₂ClCO₂H) and dichloroethanoic acid (CHCl₂CO₂H). In preferred embodiments, the short chain carboxylic acid is selected from the list consisting of formic acid, acetic acid, propionic acid, butyric acid. Another group of keratolytic agents include phenol and substituted phenolic compounds. Such compounds are known to dissolve and loosen the intracellular matrix of the hyperkeratinized tissue. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic. Yet, another class of preferred keratolytic agents includes urea and derivatives thereof. Urea possesses both keratolytic and skin-hydration properties which are beneficial to the damaged tissue of the skin.

In other embodiment, the keratolytic agent used within the composition of this invention and method of use thereof is selected from the group consisting of benzoyl peroxide, coal tar, dithranol, salicylic acid, selenium disulfide, alpha-hydroxy acid, urea, lactic acid, boric acid, retinoic acid, sodium thioglycolate, allantoin, zinc pyrithione, zinc L-pyrrolidone carboxylate, seleocysteine, selenomethionine, captopril, zofenopril, tiopronin, penicillamine, L-cysteine, gluthatione, dithiothreitol, thi orphan, cysteamine, bucillamine, dimercaprol, 1,1-ethanedi thiol, dimercaptosuccinic acid, furan-2-ylmethanethiol, omapatrilat, ovothiol A, rentiapril, thiosalicylic acid, tixocortol, mycothiol, coenzyme A, coenzyme B, disulfiram, psammaplin A, dixanthogen, pantethine, fursultiamine, octotiamine, sulbutiamme, prosultiamine, thiram, lipoic acid, lenthionine, ajoene, allicin, gemopatrilat, thioethanol, thiophospholipid, thiocholesterol, 12-mercaptododecanoic acid, 23-(9-mercaptononyl)-3,6,9,12,15,18,21-heptaoxatricosanoic Acid, and sulfanegen.

In one embodiment, the keratolytic agent within the composition of this invention and methods of use thereof is in a concentration of between 0.1% w/w to about 40% w/w of said composition.

In some embodiments, the topical composition further comprises at least one solvent. In one embodiment, the at least one solvent is selected from DMSO (dimethylsulfoxide), ethanol, isopropyl alcohol, propylene glycol, dimethyl isosorbide, isopropyl myristate, oleic acid, a polyethylene glycol, hexylene glycol, glycerin, glycofurol and combinations thereof. Each possibility represents a separate embodiment of this invention.

In some embodiments, the topical composition further comprises at least one additional active agent. In some embodiments, the topical composition further comprises at least one additional first active agent (in combination with the EGFR inhibitor; or in combination with the EGFR inhibitor and the at least second active agent) and the first active agent, or as combination with the EGFR inhibitor) selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, from about 0.5% to about 10% w/w. Each possibility represents a separate embodiment of this invention.

In some embodiments, the topical composition further comprises at least one additional second active agent (in combination with the EGFR inhibitor; or in combination with the EGFR inhibitor and the at least first active agent) selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3% or from about 3% to about 5% w/w. Each possibility represents a separate embodiment of this invention.

In some embodiments, the at least one additional first active agent is tapinarof, in a concentration of from about 0.01% to about 1%, from about 1% to about 3%, from about 3% to about 5% w/w. In some embodiments, the topical composition further comprises at least one ingredient selected from a moisturizer, a skin barrier, urea, ammonium lactate and combinations thereof, in a concentration of from about 0.01% w/w to about 1% w/w, from about 1% w/w to about 3% w/w or from about 3% w/w to about 5% w/w.

In some embodiments, the at least one EGFR inhibitor is selected from erlotinib, gefitinib, lapatinib, their salts, hydrates or solvates and combinations thereof. In one embodiment, the at least one EGFR inhibitor is erlotinib hydrochloride.

Exemplary dosages, strengths and concentrations of erlotinib, or erlotinib hydrochloride in the topical compositions of this invention, are in the range of from about or at 0.1% w/w, 0.5% w/w, 0.75% w/w, 1% w/w 1.5% w/w, 2% w/w, 2.5% w/w, 3% w/w, 3.5% w/w, 4% w/w, 4.5% w/w, 5% w/w, 5.5% w/w, 6% w/w 7% w/w, 8% w/w, 9% w/w, 10% w/w, 15% w/w 20% w/w or any ranges thereof of erlotinib, or erlotinib hydrochloride.

In some embodiments, the at least one PDE4 inhibitor is selected from roflumilast, apremilast, piclamilast, ibudliast, cilomilast their salts, hydrates or solvates and combinations thereof.

In some embodiments, the Janus kinase inhibitor (JAK inhibitor) is selected from tofacitinib, abrocitinib, ruxolitinib, delgocitinib, oclacitinib, baricitinib, peficitinib, or salt thereof and combinations thereof. In another embodiment, the Janus kinase inhibitor (JAK inhibitor) is tofacitinib or salt thereof. In another embodiment, the Janus kinase inhibitor (JAK inhibitor) is tofacitinib citrate.

Some of the above additional active agents, selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, play the role of avoiding, preventing or alleviating the EGFR inhibitor cutaneous side-effects.

According to some embodiments, the topical composition of this invention is a cream, an ointment, a gel, a lotion, a spray, a shampoo, a patch or a foam.

In one embodiment, the at least one EGFR inhibitor is erlotinib hydrochloride and the composition is formulated as a topical gel.

In one embodiment, the at least one EGFR inhibitor is erlotinib hydrochloride and the composition is formulated as an ointment.

In one embodiment, there is provided a topical composition for treatment, prevention or alleviation of a hyperkeratinization disorder in a patient in need thereof, comprising about 0.75% w/w erlotinib hydrochloride and from about 10% to about 98% w/w at least one penetration enhancer, or at least one keratolytic agent, or combination thereof, where the composition is formulated as a gel. In another embodiment, the composition comprises about 5% w/w erlotinib hydrochloride, about 63 wt % PEG 400, about 25 wt % PEG 3350 about 4 wt % about 0.5 wt % 2-phenoxyethanol and about 0.25 wt % methylparaben and the composition is formulated as an ointment. In another embodiment, the composition comprises between 0.1%-40% w/w keratolytic agent.

In one embodiment, there is provided a topical composition for treatment, prevention or alleviation of psoriasis in a patient in need thereof, comprising about 5% w/w erlotinib hydrochloride and from about 10% to about 98% w/w at least one penetration enhancer, or at least one keratolytic agent, or combination thereof, where the composition is formulated as an ointment. In another embodiment, the composition comprises about 5 wt % w/w erlotinib hydrochloride, about 63 wt % PEG 400, about 25 wt % PEG 3350 about 4 wt % about 0.5 wt % 2-phenoxyethanol and about 0.25 wt % methylparaben and the composition is formulated as an ointment. In another embodiment, the composition comprises between 0.1%-40% w/w keratolytic agent.

In one embodiment, there is provided a topical composition for treatment, prevention or alleviation of a hyperkeratinization disorder in a patient in need thereof, comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w or from about 3% w/w to about 5% w/w tapinarof and from about 10% to about 98% w/w at least one penetration enhancer, or at least one keratolytic agent, or combination thereof. In another embodiment, the composition comprises between 0.1%-40% w/w keratolytic agent.

In one embodiment, there is provided a topical composition for treatment, prevention or alleviation of a hyperkeratinization disorder in a patient in need thereof, comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w or from about 3% w/w to about 5% w/w tofacitinib citrate and from about 10% w/w to about 98% w/w at least one penetration enhancer, or at least one keratolytic agent, or combination thereof. In another embodiment, the composition comprises between 0.1%-40% w/w keratolytic agent.

In one embodiment, there is provided a topical composition for treatment, prevention or alleviation of a hyperkeratinization disorder in a patient in need thereof, comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5%, from 4% to about 6% w/w, w/w or from about 5% w/w to about 10% w/w erlotinib hydrochloride, from about 0.01% w/w to about 1% w/w, from about 1% w/w to about 3% w/w or from about 3% w/w to about 5% w/w apremilast and from about 10% w/w to about 98% w/w at least one penetration enhancer or at least one keratolytic agent, or combination thereof. In another embodiment, the composition comprises between 0.1%-40% w/w keratolytic agent.

In one embodiment, there is provided a topical composition for use in the treatment, prevention or alleviation of psoriasis, wherein the topical composition comprises from about from about 0.5% w/w to about 3% w/w, from about 3% w/w to about 5% w/w, from about 5% w/w to about 10% w/w, or from about 10% w/w to about 20% w/w erlotinib hydrochloride, from about 0.01% w/w to about 1% w/w, from about 1% w/w to about 3% w/w, from about 3% w/w to about 5% w/w tapinarof and from about 10% to about 98% w/w at least one penetration enhancer, or at least one keratolytic agent, or combination thereof. In another embodiment, the composition comprises between 0.1%-40% w/w keratolytic agent.

In one embodiment, there is provided a topical composition for use in the treatment, prevention or alleviation of psoriasis, wherein the topical composition comprises from about 0.5% w/w to about 3% w/w, from about 3% w/w to about 5% w/w, from about 5% w/w to about 10% w/w, or from about 10% w/w to about 20% w/w erlotinib hydrochloride , from about 0.01% w/w to about 1% w/w, from about 1% w/w to about 3% w/w or from about 3% w/w to about 5% w/w tofacitinib citrate and from about 10% to about 98% w/w at least one penetration enhancer, or at least one keratolytic agent, or combination thereof. In another embodiment, the composition comprises between 0.1%-40% w/w keratolytic agent.

In one embodiment, there is provided a topical composition for use in the treatment, prevention or alleviation of psoriasis, wherein the topical composition comprises from about 0.5% w/w to about 3%, from about 3% w/w to about 5% w/w, from about 5% w/w to about 10% w/w, or from about 10% w/w to about 20% w/w erlotinib hydrochloride, from about 0.01% w/w to about 1% w/w roflumilast and from about 10% w/w to about 98% w/w at least one penetration enhancer, or at least one keratolytic agent, or combination thereof. In another embodiment, the composition comprises between 0.1%-40% w/w keratolytic agent.

In some embodiments, this invention is directed to a topical composition comprising erlotinib or a pharmaceutically acceptable salt thereof in a concentration in a concentration of from about 0.1% w/w to about 20% w/w, from about 0.1% w/w to about 1% w/w, from about 1% w/w to about 2% w/w, from about 2% w/w to about 3% w/w, from about 3% w/w to about 4% w/w, from about 4% w/w to about 5% w/w, from about 3.5% to about 7.5% w/w, from about 5% w/w to about 6% w/w, from about 6% w/w to about 7% w/w, from about 7% w/w to about 8% w/w, from about 8% w/w to about 9% w/w, from about 9% w/w to about 10% w/w, from about 10% w/w to about 15% w/w, from about 15% w/w to about 20% w/w, from about 0.5% w/w to about 6% w/w, from about 0.75% w/w to about 5.5% w/w, from about 0.75% w/w to about 6% w/w, from about 0.75% w/w to about 10% w/w, from about 3.5% w/w to about 5% w/w, from about 3.5% w/w to about 5.5% w/w, from about 3.5% w/w to about 6% w/w, from about 2% w/w to about 6% w/w, from about 3% w/w to about 6% w/w, from about 3% w/w to about 7% w/w, from about 0.75% w/w to about 10% w/w, from about 5% w/w to about 10% w/w, or from about 10% w/w to about 20% w/w.

In some embodiments, the topical composition of this invention, comprises erlotinib or a pharmaceutically acceptable salt thereof in a concentration of about 0.1%, about 0.2%, about 0.25%, about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20%.

In one embodiment, this invention is directed to a topical composition comprising erlotinib or a pharmaceutically acceptable salt thereof in a concentration of from about 3.5% to about 7.5% w/w, water in a concentration of about 0% w/w to about 4% w/w, and a pharmaceutically acceptable carrier.

In one embodiment, this invention is directed to a topical composition comprising erlotinib or a pharmaceutically acceptable salt thereof in a concentration in a concentration of from about 0.1% w/w to about 20% w/w, from about 0.1% w/w to about 1% w/w, from about 1% w/w to about 2% w/w, from about 2% w/w to about 3% w/w, from about 3% w/w to about 4% w/w, from about 4% w/w to about 5% w/w, from about 3.5% to about 7.5% w/w, from about 5% w/w to about 6% w/w, from about 6% w/w to about 7% w/w, from about 7% w/w to about 8% w/w, from about 8% w/w to about 9% w/w, from about 9% w/w to about 10% w/w, from about 10% w/w to about 15% w/w, from about 15% w/w to about 20% w/w, from about 0.5% w/w to about 6% w/w, from about 0.75% w/w to about 5.5% w/w, from about 3.5% w/w to about 5.5% w/w, from about 3.5% w/w to about 6% w/w, from about 2% w/w to about 6% w/w, from about 3.5% w/w to about 6% w/w, from about 3% w/w to about 6% w/w, from about 3% w/w to about 7% w/w, from about 0.75% w/w to about 6% w/w, from about 0.75% w/w to about 10% w/w, from about 2% w/w to about 6% w/w, from about 3.5% w/w to about 5% w/w, from about 3% w/w to about 6% w/w, from about 0.75% w/w to about 10% w/w, from about 5% w/w to about 10% w/w, or from about 10% w/w to about 20% w/w, water in a concentration from about 0% w/w to about 4% w/w, and a pharmaceutically acceptable carrier. In another embodiment, the water is in a concentration from about 0% w/w to about 4% w/w, from about 0% to about 0.1% w/w, from about 0% to about 2.5% w/w, from about 0.1% to about 1% w/w, from about 0.1% to about 2%, from about 2% to about 3%, from about 3% to about 4% w/w, from 0% to about 3% w/w, from about 1.5% to about 3% w/w w/w, from about 1.75% to about 3% w/w w/w, or from about 1.75% to about 3% w/w of the composition. In another embodiment, the water is in a concentration of about 0% w/w, about 0.1% w/w, about 0.2% w/w, about 0.5% w/w, about 1% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, or about 4% w/w.

In one embodiment, this invention is directed to a topical composition comprising erlotinib or a pharmaceutically acceptable salt thereof in a concentration of from about 0.1% w/w to about 20% w/w, from about 0.1% w/w to about 10% w/w, from about 0.5% w/w to about 6% w/w, from about 0.75 to about 5.5% w/w, from about 3.5% w/w to about 5% w/w, from about 3.5% w/w to about 5.5% w/w, from about 3.5% to about 7.5% w/w, from about 3% w/w to about 6% w/w, from about 5% w/w to about 10% w/w, or from about 10% w/w to about 20% w/w water in a concentration of about 0% w/w to about 4% w/w, and a pharmaceutically acceptable carrier.

In one embodiment, the water concentration is below 0.1%, below 0.2% below 0.3%, below 0.4%, below 0.5%, below 0.6%, below 0.7%, below 0.8%, below 0.9%, below 1%, below 1.1%, below 1.2%, below 1.3%, below 1.4%, below 1.5%, below 1.6%, below 1.7%, below 1.8%, below 1.9%, below 2%, below 2.1%, below 2.2%, below 2.3%, below 2.4%, below 2.5%, below 2.6%, below 2.7%, below 2.8%, below 2.9%, below 3%, below 3.1%, below 3.2%, below 3.3%, below 3.4%, below 3.5%, below 3.6%, below 3.7%, below 3.8%, below 3.8%, below 3.9%, or below 4%. Each represents a separate embodiment of this invention.

In some embodiments, provided herein a topical composition comprising erlotinib or a pharmaceutically acceptable salt thereof in a concentration of about 3.5%-7.5% w/w, and a pharmaceutically acceptable carrier, wherein administration of said composition does not induce or induces reduced cutaneous side-effects as compared with systemic administration of the same amount of erlotinib or pharmaceutically acceptable salt thereof and wherein said administration does not induce cutaneous toxicity or induces reduced cutaneous toxicity of erlotinib or pharmaceutically acceptable salt thereof. In another embodiment, the topical composition has a systemic absorption of less than 100 ng/mL of erlotinib or less than 10 ng/mL of erlotinib metabolite (Desmethyl Erlotinib) in the plasma. In another embodiment, the administration has a systemic absorption of less than 1% or less than 0.5% by weight of erlotinib or pharmaceutically acceptable salt thereof. In other embodiments, the topical composition has a systemic absorption of between 95 ng/mL and 100 ng/mL of erlotinib or pharmaceutically acceptable salt thereof in the plasma. In other embodiments, the topical composition has a systemic absorption of between 8 and 10 ng/mL of the erlotinib metabolite (Desmethyl Erlotinib) in the plasma.

In one embodiment, this invention is directed to a topical composition comprising erlotinib or a pharmaceutically acceptable salt thereof in a concentration of about 3.5% w/w, about 5% w/w, about 5.5% w/w or about 7% w/w, water in a concentration of about 0% w/w to about 4% w/w, and a pharmaceutically acceptable carrier.

In one embodiment, this invention is directed to a topical composition comprising erlotinib or a pharmaceutically acceptable salt thereof in a concentration of from about 3.5% to about 7.5% w/w, one or more gelling agent in an amount of about 0.1% w/w to about 6% w/w of the topical composition. In other embodiments, the topical composition has a systemic absorption of less than 100 ng/mL of erlotinib or less than 10 ng/mL of erlotinib metabolite. In other embodiments, the topical composition has a systemic absorption of less than 1% or less than 0.5% by weight of erlotinib or pharmaceutically acceptable salt thereof.

In one embodiment, this invention is directed to a topical composition comprising erlotinib or a pharmaceutically acceptable salt thereof in a concentration of from about 3.5% to about 7.5% w/w, one or more gelling agent in an amount of about 0.1% w/w to about 6% w/w of the topical composition, wherein the composition is anhydrous. In other embodiments, the topical composition has a systemic absorption of less than 100 ng/mL of erlotinib or less than 10 ng/mL of erlotinib metabolite. In other embodiments, the topical composition has a systemic absorption of less than 1% or less than 0.5% by weight of erlotinib or pharmaceutically acceptable salt thereof.

In one embodiment, this invention is directed to a topical composition comprising erlotinib or a pharmaceutically acceptable salt thereof in a concentration of from about 3.5% w/w to about 5% from about 3.5% w/w to about 5.5% w/w, from about 3% w/w to about 6% w/w, from about 3.5% to about 7.5% w/w, and a pharmaceutically acceptable carrier, wherein the composition further comprising one or more gelling agent in an amount of about 0.1% w/w to about 6% w/w of the topical composition, and wherein the composition is anhydrous. In other embodiments, the topical composition has a systemic absorption of less than 100 ng/mL of erlotinib or less than 10 ng/mL of erlotinib metabolite. In other embodiments, the topical composition has a systemic absorption of less than 1% or less than 0.5% by weight of erlotinib or pharmaceutically acceptable salt thereof.

In some embodiments, the one or more gelling agent is present from about 0.1% w/w to about 6% w/w of the topical composition, about 0.1% w/w to about 5% w/w of the topical composition, about 0.1% w/w to about 4% w/w of the topical composition, about 0.1% w/w to about 3% w/w of the topical composition, about 0.1% w/w to about 2% w/w of the topical composition, about 2% w/w to about 3% w/w of the topical composition, about 3% w/w to about 4% w/w of the topical composition, or about 0.1% w/w to about 1% w/w of the topical composition. In some embodiments, the gelling agent is present at about 1% w/w, about 2% w/w, about 3% w/w, about 4% w/w, about 5% w/w, about 6% w/w, about 0.5% w/w, about 1.5% w/w, about 2.5% w/w, about 3.5% w/w, about 4.5% w/w, or about 5.5% w/w. In one embodiment, the gelling agent is present at about 3% w/w. In other embodiments, the topical composition has a systemic absorption of less than 100 ng/mL of erlotinib or less than 10 ng/mL of erlotinib metabolite. In other embodiments, the topical composition has a systemic absorption of less than 1% or less than 0.5% by weight of erlotinib or pharmaceutically acceptable salt thereof.

In some embodiments, the gelling agent is selected from poloxamers, carbomers, or mixtures thereof.

In some embodiments, the poloxamer is selected from poloxamer P-188, poloxamer P-138, poloxamer P-237, poloxamer P-288, poloxamer P-124, poloxamer P338, poloxamer P-407, poly(ethylene glycol/DL-lactide-Co-glyceride) poly(caprolactum), hydroxypropyl cellulose (KLUCEL®), glyceryl tris 12-hydroxy stearate, hydroxy stearin, hydroxypropyl cellulose (HPC), propylene carbonate, polyvinyl pyrrolidine, or mixtures thereof.

In some embodiments, the carbomers is selected from carbomer 981, carbomer 980, carbomer 934, carbomer 934P, carbomer 940, carbomer 941, carbomer 1342, polycarbophil, calcium polycarbophil, or mixtures thereof.

In one embodiment, the gelling agent is Carbopol 980.

In one embodiment, this invention is directed to a topical composition comprising erlotinib or a pharmaceutically acceptable salt thereof in a concentration of about 0.1% w/w to about 20% w/w, water in a concentration of about 0% w/w to about 4% w/w, and a pharmaceutically acceptable carrier, wherein the topical composition is formulated as a cream, an ointment, a gel, a spray, a shampoo, a skin patch, a mucosal patch, a solution, a lotion or a foam. In another embodiment, the topical composition is formulated as a cream. In another embodiment, the topical composition is formulated as an ointment. In another embodiment, the topical composition is formulated as a gel. In another embodiment, the topical composition is formulated as a spray. In another embodiment, the topical composition is formulated as a shampoo. In another embodiment, the topical composition is formulated as a skin patch. In another embodiment, the topical composition is formulated as a mucosal patch. In another embodiment, the topical composition is formulated as a solution. In another embodiment, the topical composition is formulated as a lotion. In another embodiment, the topical composition is formulated as a foam. In other embodiments, the topical composition has a systemic absorption of less than 100 ng/mL of erlotinib or less than 10 ng/mL of erlotinib metabolite. In other embodiments, the topical composition has a systemic absorption of less than 1% or less than 0.5% by weight of erlotinib or pharmaceutically acceptable salt thereof.

In one embodiment, this invention is directed to a topical composition comprising erlotinib or a pharmaceutically acceptable salt thereof in a concentration of from about 3.5% to about 7.5% w/w, water in a concentration of about 0% w/w to about 4% w/w, and a pharmaceutically acceptable carrier, wherein the topical composition is formulated as an ointment or a gel. In other embodiments, the topical composition has a systemic absorption of less than 100 ng/mL of erlotinib or less than 10 ng/mL of erlotinib metabolite. In other embodiments, the topical composition has a systemic absorption of less than 1% or less than 0.5% by weight of erlotinib or pharmaceutically acceptable salt thereof.

In one embodiment, this invention is directed to a topical composition for use in the treatment, prevention or alleviation of a non-melanoma skin cancer selected from the group consisting of basal cell carcinoma (BCC), squamous cell skin cancer (SCC), actinic keratosis, Gorlin syndrome, Merkel cell carcinoma, Bowen's disease, classic Kaposi sarcoma, hereditary leiomyomatosis (Reed'ds syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma, wherein the composition administered to a subject in need, comprises erlotinib or a pharmaceutically acceptable salt thereof in a concentration of about 0.1% w/w to about 20% w/w. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a topical composition for use in the treatment, prevention or alleviation of a non-melanoma skin cancer selected from the group consisting of basal cell carcinoma (BCC), squamous cell skin cancer (SCC), actinic keratosis, Gorlin syndrome, Merkel cell carcinoma, Bowen's disease, classic Kaposi sarcoma, hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma, wherein the composition comprises erlotinib or a pharmaceutically acceptable salt thereof in a concentration of about from about 3.5% to about 7.5% w/w, water in a concentration of about 0% w/w to about 4% w/w, and a pharmaceutically acceptable carrier. Each represents a separate embodiment of this invention.

Non-limiting examples of skin or a mucosal disorder that may be treated by the topical compositions of this invention include basal cell carcinoma (BCC), squamous cell skin cancer (SCC), actinic keratosis, Gorlin syndrome, Merkel cell carcinoma, Bowen's disease, classic Kaposi sarcoma, hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, sebaceous carcinoma, keratinization skin disorder, hidradenitis suppurativa, prurigo nodularis, prurigo pigmentosa, Darier's disease, Hailey-Hailey disease, palmoplantar keratoderma, or pachyonychia congenita. Each represents a separate embodiment of this invention.

In one embodiment, this invention is directed to a topical composition for use in the treatment, prevention or alleviation of a skin or a mucosal disorder, wherein the skin or mu selected from the group consisting of basal cell carcinoma (BCC), squamous cell skin cancer (SCC), actinic keratosis, Gorlin syndrome, Merkel cell carcinoma, Bowen's disease, classic Kaposi sarcoma, hereditary leiomyomatosis (Reed'ds syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, sebaceous carcinoma, keratinization skin disorder, hidradenitis suppurativa, prurigo nodularis or prurigo pigmentosa, wherein the composition comprises erlotinib or a pharmaceutically acceptable salt thereof in a concentration of about 0.1% w/w to about 20% w/w, water in a concentration of about 0% w/w to about 4% w/w, and a pharmaceutically acceptable carrier. In another embodiment, the keratinization skin disorders are selected from Darier's disease, Hailey-Hailey disease, palmoplantar keratoderma, or pachyonychia congenita. In another embodiment, the skin or a mucosal disorder is keratinization skin disorder. In another embodiment, the skin or a mucosal disorder is hidradenitis suppurativa. In another embodiment, the skin or a mucosal disorder is prurigo nodularis. In another embodiment, the skin or a mucosal disorder is prurigo pigmentosa. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a topical composition for use in the treatment, prevention or alleviation of a skin or a mucosal disorder a selected from the group consisting of basal cell carcinoma (BCC), squamous cell skin cancer (SCC), actinic keratosis, Gorlin syndrome, Merkel cell carcinoma, Bowen's disease, classic Kaposi sarcoma, hereditary leiomyomatosis (Reed'ds syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, sebaceous carcinoma, keratinization skin disorder, hidradenitis suppurativa, prurigo nodularis, prurigo pigmentosa, wherein the composition administered to a subject in need, comprises erlotinib or a pharmaceutically acceptable salt thereof in a concentration of about 0.1% w/w to about 20% w/w. In other embodiments, the subject does not suffer from a skin keratinization disorder that requires concomitant treatment of the skin keratinization disorder. In another embodiment, the skin keratinization disorder is genetic skin keratinization disorder.

In another embodiment, the keratinization skin disorders are selected from Darier's disease, Hailey-Hailey disease, palmoplantar keratoderma, or pachyonychia congenita. In another embodiment, the skin or a mucosal disorder is keratinization skin disorder. In another embodiment, the skin or a mucosal disorder is hidradenitis suppurativa. In another embodiment, the skin or a mucosal disorder is prurigo nodularis. In another embodiment, the skin or a mucosal disorder is prurigo pigmentosa.

Pharmaceutical acceptable carriers or vehicles suitable for preparation of the compositions provided herein include any such carriers known to those skilled in the art to be suitable for the particular mode of administration.

Pharmaceutical acceptable carriers or vehicles suitable for administration of erlotinib provided herein include any such carriers known to those skilled in the art to be suitable for the particular mode of administration. In addition, the erlotinib may be formulated as the sole pharmaceutically active agent in the composition or may be combined with other active agents. The erlotinib included in the carrier in an amount sufficient to exert a therapeutically useful effect. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application are those suited for use including: creams, gels, ointments, emulsions, solutions, elixirs, lotions, suspensions, tinctures, pastes, foams, aerosols, sprays, and patches. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

In some embodiments, the “pharmaceuticaly acceptable salt” of erlotinib is an acid salt forms can be created from acids including aceturic, 4-acetamido-benzoic, adipic, aminohippuric, 4-amino-salicylic, alginic, aspartic, boric, butyric, capric (decanoic), caproic (hexanoic), carbonic, camphoric, camphorsulfonic, caprylic (octanoic), cyclamic, cinnamic, 2,2-dichloro-acetic, di(t-butyl)-naphthalenesulfonic, di(t-butyl)-naphthalenedisulfonic, dehydroacetic, diatrizoic, dodecylsulfuric, ethane-1,2-disulfonic, edetic, ethanesulfonic, 2-ethyl-hexanoic, erythorbic, formic, fumaric, galactaric (mucic), gentisic, glucoheptanoic, gluconic, glucuronic, glutamic, glutaric, glycerophosphoric, glycolic, hippuric, hydrochloric, hydrobromic, hydroiodic, 2-(4-hydroxybenzoyl)-benzoic, 2-hydroxy-ethanesulfonic (isethionic), 1-hydroxy-2-naphtoic, isobutyric, lactic, lactobionic, lauric, iodoxamic, isostearic, maleic, malic, malonic, mandelic, medronic, methanesulfonic, methaphosphoric, methylboronic, myristic, naphthalene-1,5-disulfonic, naphthalene-2-sulfonic, nicotinic, oleic, oxalic, palmitic, pentetic, propionic, propanoic, pyroglutamic, pyruvic, phosphoric, sebacic, sorbic, stearic (octadecanoic), suberic, succinic, sulfuric, tartaric, thiazoximic, thiocyanic, toluenesulfonic, trifluoroacetic and undecylenic (undec-10-enoic) acids. Each possibility represents a separate embodiment of the present invention.

In some embodiments, there is provided an injectable composition for treatment, prevention or alleviation of a skin or mucosal disorder selected from the group consisting of psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa comprising at least one EGFR inhibitor selected from the group consisting of erlotinib, gefitinib, lapatinib, cetuximab, panitumumab, vandetanib, necitumumab, osimertinib and combinations thereof.

In another embodiment, the concentration of the EGFR inhibitor in the injectable composition is from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w.

In one embodiment, the injectable composition further comprises at least one solvent as described hereinabove.

In one embodiment, the injectable composition further comprises at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, from about 0.5% to about 10% w/w.

In one embodiment, the injectable composition further comprises at least one additional second active agent (in addition to the EGFR inhibitor and the first active agent, or as combination with the EGFR inhibitor) co selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3% or from about 3% to about 5% w/w.

In some embodiments, the injectable composition further comprises (i) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w; and (ii) comprises at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3% or from about 3% to about 5% w/w.

In some embodiments, there is provided a method of treatment, prevention or alleviation of a skin or mucosal disorder selected from the group consisting of psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of a therapeutically effective amount of the composition as described hereinabove. In one embodiment, the composition comprises at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from about 5% to about 10% w/w, from 4% to about 6% w/w, or from about 10% to about 20% w/w. In one embodiment, the composition is topical.

In some embodiments, this invention provides a method of treatment, prevention or alleviation of psoriasis, by topical or injectable administration to a subject in need thereof a composition comprising erlotinib or pharmaceutically acceptable salt thereof in a concentration of from about 0.5% to about 20% w/w. In other embodiments, the method comprises daily, twice daily or three times per day topical application of therapeutically effective amounts of erlotinib or pharmaceutically acceptable salt thereof to the skin portion of the subject affected by psoriasis until the skin or mucosal disorder is cured, prevented or alleviated or according to doctor's instructions.

In other embodiments, this invention provides a method of treatment, prevention or alleviation of psoriasis, by topical or injectable administration to a subject in need thereof a composition comprising erlotinib or pharmaceutically acceptable salt thereof in a concentration of from about 0.5% to about 20% w/w, wherein said method does not induce cutaneous toxicity, or induces reduced cutaneous toxicity of erlotinib or pharmaceutically acceptable salt thereof. In other embodiments, this invention provides a method of treatment, prevention or alleviation of psoriasis, by topical or injectable administration to a subject in need thereof a composition comprising erlotinib or pharmaceutically acceptable salt thereof in a concentration of from about 0.5% to about 20% w/w, wherein said method does not induce cutaneous toxicity, or induces reduced cutaneous toxicity of erlotinib or pharmaceutically acceptable salt thereof compared to erlotinib or pharmaceutically acceptable salt thereof administered systemically. In other embodiment, the psoriasis is palmoplantar psoriasis. In other embodiments, the method comprises daily, twice daily or three times per day topical application of therapeutically effective amounts of erlotinib or pharmaceutically acceptable salt thereof to the skin portion of the subject affected by psoriasis until the skin or mucosal disorder is cured, prevented or alleviated or according to doctor's instructions.

In other embodiments, this invention provides a method of treatment, prevention or alleviation of psoriasis, by topical or injectable administration to a subject in need thereof a composition comprising erlotinib or pharmaceutically acceptable salt thereof in a concentration of from about 0.5% to about 3% w/w, from about 3% w/w to about 5% w/w, from 4% to about 6% w/w, from about 5% w/w to about 10% w/w, or from about 10% w/w to about 20% w/w erlotinib or pharmaceutically acceptable salt thereof and from about 10% to about 98% w/w at least one penetration enhancer, or at least one keratolytic agent, or combination thereof. In another embodiment, the composition comprises between 0.1%-40% w/w keratolytic agent. In other embodiments, the psoriasis is palmoplantar psoriasis. In other embodiments, the method comprises daily, twice daily or three times per day topical application of therapeutically effective amounts of the said composition to the skin portion of the subject affected by psoriasis until the skin or mucosal disorder is cured, prevented or alleviated or according to doctor's instructions.

In other embodiments, this invention provides a method of treatment, prevention or alleviation of psoriasis, by topical or injectable administration to a subject in need thereof a composition comprising erlotinib or pharmaceutically acceptable salt thereof in a concentration of from about 0.5% to about 3% w/w, from about 3% w/w to about 5% w/w, from 4% to about 6% w/w, from about 5% w/w to about 10% w/w, or from about 10% w/w to about 20% w/w erlotinib or pharmaceutically acceptable salt thereof and from about 10% to about 98% w/w at least one penetration enhancer, or at least one keratolytic agent, or combination thereof; wherein the method does not induce or induces reduced cutaneous side-effects as compared with systemic administration of the same amount of erlotinib or pharmaceutically acceptable salt thereof and wherein said method does not induce cutaneous toxicity, or induces reduced cutaneous toxicity of erlotinib or pharmaceutically acceptable salt thereof compared to erlotinib or pharmaceutically acceptable salt thereof administered systemically. In other embodiments, the psoriasis is palmoplantar psoriasis. In other embodiments, the method comprises daily, twice daily or three times per day topical application of therapeutically effective amounts of the said composition to the skin portion of the subject affected by psoriasis until the skin or mucosal disorder is cured, prevented or alleviated or according to doctor's instructions.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof
(i) a composition comprising a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w; and
(ii) a composition comprising at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, from about 0.5% to about 10% w/w, wherein the two separate compositions are administered concomitantly or sequentially, in either order.

In one additional embodiment, this invention is directed to a method of treatment, prevention or alleviation of psoriasis, by topical or injectable administration to a subject in need thereof
a composition comprising:
(i) a therapeutically effective amount of erlotinib or pharmaceutically acceptable salt thereof, in a concentration of 0.5% to about 20% w/w; and
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, from about 0.5% to about 10% w/w. In other embodiments, the psoriasis is palmoplantar psoriasis.

In other embodiment, said erlotinib and said at least one additional first active agent exhibit an additive or synergistic effect.

In some embodiments, the compositions are topical compositions.

In one additional embodiment, this invention is directed to a method of treatment, prevention or alleviation of psoriasis, by topical or injectable administration to a subject in need thereof
(i) a composition comprising a therapeutically effective amount of erlotinib or pharmaceutically acceptable salt thereof, in a concentration of 0.5% to about 20% w/w;

and
(ii) a composition comprising a therapeutically effective amount of at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w. In other embodiment, the psoriasis is palmoplantar psoriasis.

In another embodiment, the method comprises administering a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w, wherein the two separate compositions are administered concomitantly or sequentially, in either order.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of
a composition comprising:
(i) a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 10%, from about 1% to about 20%, from about 0.01% to about 1%, from about 1% to about 3%, from about 3% to about 5% w/w, from about 5% to about 10% w/w, from 4% to about 6% w/w, or from about 10% to about 20% w/w; and
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, from about 0.5% to about 10% w/w.

In some embodiments, the composition is a topical composition.

In another embodiment, the method further comprises administering a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of
(i) a composition comprising a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w; and
(ii) a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof; in a concentration of from about 0.01% to about 1%, from about 1% to about 3%, from about 3% to about 5% w/w,
   wherein the two separate compositions are administered concomitantly or sequentially, in either order. In some embodiments, the compositions are topical compositions.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of
a composition comprising:
(i) a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w; and
(ii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof; in a concentration of from about 0.01% to about 1%, from about 1% to about 3%, from about 3% to about 5% w/w.

In one additional embodiment, this invention is directed to a method of treatment, prevention or alleviation of psoriasis, by topical or injectable administration to a subject in need thereof
a composition comprising:
(i) a therapeutically effective amount of erlotinib or pharmaceutically acceptable salt thereof, in a concentration of from 0.5% to about 20% w/w; and
(ii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w. In other embodiment, the psoriasis is palmoplantar psoriasis. In other embodiment, said erlotinib and said at least one additional second active agent exhibit an additive or synergistic effect.

In some embodiments, the composition is a topical composition.

In another embodiment, the method further comprises administering a composition comprising at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, from about 0.5% to about 10% w/w.

In one additional embodiment, this invention is directed to a method of treatment, prevention or alleviation of psoriasis, by topical or injectable administration to a subject in need thereof
(i) a composition comprising a therapeutically effective amount of erlotinib or pharmaceutically acceptable salt thereof, in a concentration of from 0.5% to about 20% w/w; and
(ii) a composition comprising a therapeutically effective amount of at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w. In other embodiment, the psoriasis is palmoplantar psoriasis. In other embodiment, said erlotinib and said at least one additional second active agent exhibit an additive or synergistic effect.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof
(i) a composition comprising a therapeutically effective amount of at least one EGFR inhibitor selected from the group consisting of erlotinib, gefitinib, lapatinib, cetuximab, panitumumab, vandetanib, necitumumab, osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w;
(ii) a composition comprising at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, from about 0.5% to about 10% w/w; and
(iii) a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof in a concentration of from about 0.01% to about 1%, from about 1% to about 3%, from about 3% to about 5% w/w;
   wherein the three separate compositions are administered concomitantly or sequentially, in either order.

In one additional embodiment, this invention is directed to a method of treatment, prevention or alleviation of psoriasis, by topical or injectable administration to a subject in need thereof
a composition comprising:
(i) a therapeutically effective amount of erlotinib or pharmaceutically acceptable salt thereof in a concentration of from about 0.5% to about 20% w/w;
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, from about 0.5% to about 10% w/w; and
(iii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w. In other embodiments, the psoriasis is palmoplantar psoriasis.

In some embodiments, the compositions are topical compositions.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichthyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of
a composition comprising:
(i) a therapeutically effective amount of at least one EGFR inhibitor selected from the group consisting of erlotinib, gefitinib, lapatinib, cetuximab, panitumumab, vandetanib, necitumumab, osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w;
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, from about 0.5% to about 10% w/w; and
(iii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w.

In some embodiments, the composition is a topical composition.

In one additional embodiment, this invention is directed to a method of treatment, prevention or alleviation of psoriasis, by topical or injectable administration to a subject in need thereof
(i) a composition comprising a therapeutically effective amount of erlotinib or pharmaceutically acceptable salt thereof in a concentration of from about 0.5% to about 20% w/w;
(ii) a composition comprising a therapeutically effective amount of at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, from about 0.5% to about 10% w/w; and
(iii) a composition comprising a therapeutically effective amount of at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w. In other embodiments, the psoriasis is palmoplantar psoriasis.

In one embodiment, the at least one EGFR inhibitor and at least one additional first and/or second active agent within the above methods exhibit an additive or synergistic effect.

In some embodiments, the skin or mucosal disorder treated, prevented or alleviated within the methods described hereinabove is psoriasis.

In some embodiments, the skin or mucosal disorder treated, prevented or alleviated within the methods described hereinabove is palmoplantar psoriasis.

In some embodiments, the skin or mucosal disorder treated, prevented or alleviated within the methods described hereinabove is selected from a keratinization skin disorder and a keratinization mucosal disorder. In another embodiment, the keratinization skin disorder is hyperkeratinization skin disorder.

In some embodiments, this invention is directed to a method of treating, preventing or alleviating non-melanoma skin cancer, wherein the method comprises topical administration to a subject in need thereof a therapeutically effective amount of a topical composition of this invention.

In some embodiments, this invention is directed to a method of treating, preventing or alleviating non-melanoma skin cancer, wherein the method comprises topical administration to a subject in need thereof a therapeutically effective amount of a topical composition of this invention, wherein the method reduces the proliferation index (Ki-67).

In some embodiments, the non-melanoma skin cancer is skin cancer basal cell carcinoma (BCC), squamous cell skin cancer (SCC), actinic keratosis, Gorlin syndrome, Merkel cell carcinoma, Bowen's disease, classic Kaposi sarcoma, Hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DS AP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, or sebaceous carcinoma.

In one embodiment, the non-melanoma skin cancer is skin cancer basal cell carcinoma (BCC). In one embodiment, the non-melanoma skin cancer is squamous cell skin cancer (SCC). In one embodiment, the non-melanoma skin cancer is actinic keratosis. In one embodiment, the non-melanoma skin cancer is Gorlin syndrome. In one embodiment, the non-melanoma skin cancer is Merkel cell carcinoma. In one embodiment, the non-melanoma skin cancer is Bowen's disease. In one embodiment, the non-melanoma skin cancer is classic Kaposi sarcoma. In one embodiment, the non-melanoma skin cancer is Hereditary leiomyomatosis (Reed's syndrome). In one embodiment, the non-melanoma skin cancer is Ankyloblepharon-ectodermal defects-cleft lip/palate (AEC) syndrome. In one embodiment, the non-melanoma skin cancer is dermatofibrosarcoma protuberans. In one embodiment, the non-melanoma skin cancer is granular cell tumors on skin. In one embodiment, the non-melanoma skin cancer is disseminated superficial actinic porokeratosis (DSAP). In one embodiment, the non-melanoma skin cancer is Sezary syndrome. In one embodiment, the non-melanoma skin cancer is keratocystic odontogenic tumor. In one embodiment, the non-melanoma skin cancer is epidermolysis Bullosa-associated squamous cell carcinoma. In one embodiment, the non-melanoma skin cancer is angiosarcoma of the skin. In one embodiment, the non-melanoma skin cancer is cutaneous B-cell lymphoma. In one embodiment, the non-melanoma skin cancer is cutaneous T-cell lymphoma. In one embodiment, the non-melanoma skin cancer is sebaceous carcinoma.

In some embodiments, this invention is directed to a method of treating, preventing or alleviating a skin or mucosal disorder, wherein the method comprises topical administration to a subject in need thereof a therapeutically effective amount of a topical composition of this invention. In another embodiment, the skin or mucosal disorder is selected from a group consisting of non-melanoma skin cancer, skin cancer basal cell carcinoma (BCC), squamous cell skin cancer (SCC), actinic keratosis, Gorlin syndrome, Merkel cell carcinoma, Bowen's disease, classic Kaposi sarcoma, Hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, sebaceous carcinoma, keratinization skin disorder, pachyonychia congenita, hidradenitis suppurativa, prurigo nodularis or prurigo pigmentosa,. Each represents a separate embodiment of this invention. In another embodiment, the keratinization skin disorder is selected from Darier's disease, Hailey-Hailey disease, or palmoplantar keratoderma. In another embodiment, the method of treating, preventing or alleviating a skin or mucosal disorder wherein the method comprises topical administration to a subject in need thereof a therapeutically effective amount of a topical composition of this invention, reduces the proliferation index (Ki-67).

In some embodiments, this invention is directed to a method of treating, preventing or alleviating a skin or mucosal disorder, wherein the method comprises topical administration to a subject in need thereof a therapeutically effective amount of a topical composition of this invention, wherein the skin or mucosal disorder is selected from a group consisting of keratinization skin disorder, pachyonychia congenita, hidradenitis suppurativa, prurigo nodularis or prurigo pigmentosa.

In another embodiment, the skin or mucosal disorder is keratinization skin disorder. In another embodiment, the skin or mucosal disorder is pachyonychia congenita. In another embodiment, the skin or mucosal disorder is hidradenitis suppurativa. In another embodiment, the skin or mucosal disorder is prurigo nodularis. In another embodiment, the skin or mucosal disorder is prurigo pigmentosa.

In another embodiment, the keratinization skin disorder is selected from Darier's disease, Hailey-Hailey disease, or palmoplantar keratoderma. In another embodiment, the keratinization skin disorder is Darier's disease. In another embodiment, the keratinization skin disorder is Hailey-Hailey disease. In another embodiment, the keratinization skin disorder is palmoplantar keratoderma.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w. In another embodiment, the method reduces the proliferation index (Ki-67).

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, wherein said subject does not suffer from a skin keratinization disorder that requires concomitant treatment of the skin keratinization disorder wherein said subject does not suffer from a skin keratinization disorder that requires concomitant treatment of the skin keratinization disorder. In another embodiment, the skin keratinization disorder is genetic skin keratinization disorder. In another embodiment, the method reduces the proliferation index (Ki-67).

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration from about 0.1% w/w to about 20% w/w, from about 0.1% w/w to about 10% w/w, from about 0.5% w/w to about 6% w/w, from about 0.75% w/w to about 6% w/w, from about 2% w/w to about 6% w/w, from about 3% w/w to about 5% w/w from about 3.5% w/w to about 5% w/w, from about 3.5% to about 7.5% w/w, from about 3.5% w/w to about 5.5 w/w, from about 3.5% w/w to about 6% w/w, from about 3% w/w to about 6% w/w, from about 3% w/w to about 7% w/w, from about 3% w/w to about 10% w/w, from about 2% w/w to about 6% w/w, from about 4% w/w to about 6% w/w, from about 5% w/w to about 6% w/w, from about 2% w/w to about 7% w/w, from about 4% w/w to about 7% w/w, from about 4% w/w to about 8%, from about 5% w/w to about 10% w/w, from about 5% w/w to about 15% w/w, or from about 10% w/w to about 20% w/w. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 3.5% w/w, about 5% w/w, about 5.5% w/w or about 7% w/w. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration from about 0.1% w/w to about 20% w/w, from about 0.1% w/w to about 10% w/w, from about 0.5% w/w to about 6% w/w, from about 0.75% w/w to about 6% w/w, from about 3.5% w/w to about 5% w/w, from about 3.5% w/w to about 5.5% w/w, from about 3% w/w to about 7.5% w/w, from about 3% w/w to about 6% w/w, from about 5% w/w to about 10% w/w, or from about 10% w/w to about 20% w/w, wherein the non-melanoma skin cancer is selected from a group consisting of skin cancer basal cell carcinoma (BCC), squamous cell skin cancer (SCC), actinic keratosis, Gorlin syndrome, Merkel cell carcinoma, Bowen's disease, classic Kaposi sarcoma, Hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, wherein the method does not induce or induces reduced cutaneous side-effects as compared with systemic administration of the same amount of erlotinib or pharmaceutically acceptable salt thereof and wherein said method does not induce cutaneous toxicity or induces reduced cutaneous toxicity of erlotinib or pharmaceutically acceptable salt thereof. In another embodiment, the erlotinib or a pharmaceutically acceptable salt thereof is in a concentration of about 0.1% w/w to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% to about 1%, from about 1% to about 3%, from about 3.5% w/w to about 5% w/w, from about 3.5% w/w to about 5.5% w/w, from about 3.5% to about 7.5% w/w, from about 3% to about 5% w/w, from 4% to about 6% w/w, from about 0.5% w/w to about 6% w/w, from about 0.75% w/w to about 5.5% w/w, from about 3.5% w/w to about 5.5% w/w, from about 3.5% w/w to about 5% w/w, about 3% w/w to about 6% w/w, about 5% w/w to about 10% w/w, or about 10% w/w to about 20% w/w. Each represents a separate embodiment of this invention. In another embodiment, the erlotinib is in a concentration of about 3.5% w/w or about 5% or about 5.5% w/w. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, wherein the method comprises once daily, twice daily or three times per day topical application of therapeutically effective amounts of the said composition to the skin portion of the subject affected by skin or mucosal disorder is cured, prevented or alleviated or according to doctor's instructions. In another embodiment, the erlotinib or a pharmaceutically acceptable salt thereof is in a concentration of about 0.1% w/w to about 20% w/w, from about 0.1% to about 10% w/w, from about 0.5% to about 20% w/w, from about 1% to about 20% w/w, from about 0.1% to about 1%, from about 1% to about 3%, from about 3% to about 5% w/w, from about 3% w/w to about 7.5% w/w, from 4% to about 6% w/w, from about 0.5% w/w to about 6% w/w, from about 0.75% w/w to about 5.5% w/w, from about 3.5% w/w to about 5% w/w, from about 3.5% w/w to about 5.5% w/w, about 3% w/w to about 6% w/w, about 5% w/w to about 10% w/w, or about 10% w/w to about 20% w/w. In another embodiment, the erlotinib is in a concentration of about 3.5% w/w. In another embodiment, the erlotinib is in a concentration of about 5% w/w. In another embodiment, the erlotinib is in a concentration of about 5.5% w/w. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, wherein said topical composition is formulated as a cream, an ointment, a gel, a spray, a shampoo, a skin patch, a mucosal patch, a solution, a lotion or a foam. In another embodiment, wherein said topical composition is formulated as a cream. In another embodiment, wherein said topical composition is formulated as an ointment. In another embodiment, wherein said topical composition is formulated as a gel. In another embodiment, wherein said topical composition is formulated as a spray. In another embodiment, wherein said topical composition is formulated as a shampoo. In another embodiment, wherein said topical composition is formulated as a skin patch. In another embodiment, wherein said topical composition is formulated as a mucosal patch. In another embodiment, wherein said topical composition is formulated as a solution. In another embodiment, wherein said topical composition is formulated as a lotion. In another embodiment, wherein said topical composition is formulated as a foam. In another embodiment, the erlotinib or a pharmaceutically acceptable salt thereof is in a concentration of about 0.1% w/w to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from about 3% w/w to about 7.5% w/w, from 4% to about 6% w/w, from about 0.5% w/w to about 6% w/w, from about 0.75% w/w to about 5.5% w/w, from about 3% w/w to about 6% w/w, from about 3.5% w/w to about 5% w/w, from about 3.5% w/w to about 5.5% w/w, from about 5% w/w to about 10% w/w, or from about 10% w/w to about 20% w/w. In another embodiment, the erlotinib is in a concentration of about 0.75% w/w. In another embodiment, the erlotinib is in a concentration of about 3.5% w/w. In another embodiment, the erlotinib is in a concentration of about 5% w/w. In another embodiment, the erlotinib is in a concentration of about 5% w/w. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, wherein said topical composition is formulated as an ointment, or a gel. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, and water in a concentration of about 0% w/w to about 4% w/w. In another embodiment, the erlotinib or a pharmaceutically acceptable salt thereof is in a concentration of about 0.1% w/w to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from about 3.5% to about 5% w/w, from about 3.5% to about 5.5% w/w, from 4% to about 6% w/w, from about 0.5% w/w to about 6% w/w, from about 0.75% w/w to about 5.5% w/w, about 3% w/w to about 6% w/w, about 5% w/w to about 10% w/w, or about 10% w/w to about 20% w/w. In another embodiment, the erlotinib is in a concentration of about 3.5% w/w, about 5% w/w, about 5.5% w/w, or about 7% w/w. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, and water in a concentration of about 0% w/w to about 4% w/w, wherein the non-melanoma skin cancer is selected from a group consisting of skin cancer basal cell carcinoma (BCC), squamous cell skin cancer (SCC), actinic keratosis, Gorlin syndrome, Merkel cell carcinoma, Bowen's disease, classic Kaposi sarcoma, Hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, water in a concentration of about 0% w/w to about 4% w/w, and a pharmaceutically acceptable carrier. In another embodiment, the composition is anhydrous.

In one embodiment, this invention is directed to a method of treating, preventing or alleviating non-melanoma skin cancer, wherein the method comprises topical administration to a subject in need thereof a therapeutically effective amount of a topical composition compriding erlotinib or a pharmaceutically acceptable salt thereof in a concentration of about 0.1% w/w to about 20% w/w, water in a concentration of about 0% w/w to about 4% w/w, gelling agent in an amount of about 0.1% w/w to about 6% w/w of the topical composition, and a pharmaceutically acceptable carrier.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, water in a concentration of about 0% w/w to about 4% w/w, and a pharmaceutically acceptable carrier, and wherein the methos reduces the proliferation index (Ki-67). In other embodiments, the subject does not suffer from a skin keratinization disorder that requires concomitant treatment of the skin keratinization disorder. In another embodiment, the skin keratinization disorder is genetic skin keratinization disorder.In one embodiment, this invention is directed to a method of treating, preventing or alleviating non-melanoma skin cancer, wherein the method comprises administering a topical composition comprising erlotinib or a pharmaceutically acceptable salt thereof in a concentration of about 3.5%-7.5% w/w, and a pharmaceutically acceptable carrier, wherein administration of said composition does not induce or induces reduced cutaneous side-effects as compared with systemic administration of the same amount of erlotinib or pharmaceutically acceptable salt thereof and wherein said administration does not induce cutaneous toxicity or induces reduced cutaneous toxicity of erlotinib or pharmaceutically acceptable salt thereof.

In one embodiment, this invention is directed to a method of treating, preventing or alleviating non-melanoma skin cancer, wherein the method comprises administering a topical composition comprising erlotinib or a pharmaceutically acceptable salt thereof in a concentration of about 3.5%-7.5% w/w, and a pharmaceutically acceptable carrier, wherein administration of said composition does not induce or induces reduced cutaneous side-effects as compared with systemic administration of the same amount of erlotinib or pharmaceutically acceptable salt thereof and wherein said administration does not induce cutaneous toxicity or induces reduced cutaneous toxicity of erlotinib or pharmaceutically acceptable salt thereof and the topical composition has a systemic absorption of less than 100 ng/mL of erlotinib or less than 10 ng/mL of erlotinib metabolite.

In one embodiment, this invention is directed to a method of treating, preventing or alleviating non-melanoma skin cancer, wherein the method comprises administering a topical composition comprising erlotinib or a pharmaceutically acceptable salt thereof in a concentration of about 3.5%-7.5% w/w, and a pharmaceutically acceptable carrier, wherein administration of said composition does not induce or induces reduced cutaneous side-effects as compared with systemic administration of the same amount of erlotinib or pharmaceutically acceptable salt thereof and wherein said administration does not induce cutaneous toxicity or induces reduced cutaneous toxicity of erlotinib or pharmaceutically acceptable salt thereof and the topical composition has a systemic absorption of less than 1% or less than 0.5% by weight of erlotinib or pharmaceutically acceptable salt thereof.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of from about 3% w/w to about 7.5% w/w, gelling agent in an amount of about 0.1% w/w to about 6% w/w of the topical composition, and a pharmaceutically acceptable carrier, and wherein the composition is anhydrous.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of from about 3% w/w to about 7.5% w/w, and a pharmaceutically acceptable carrier, wherein the non-melanoma skin cancer is selected from a group consisting of skin cancer basal cell carcinoma (BCC), squamous cell skin cancer (SCC), actinic keratosis, Gorlin syndrome, Merkel cell carcinoma, Bowen's disease, classic Kaposi sarcoma, Hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma. In another embodiments, the composition comprises water in a concentration of about 0% w/w to about 4% w/w.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 3.5% w/w, about 5% w/w, about 5.5% w/w or about 7% w/w, water in a concentration of about 0% w/w to about 4% w/w, and a pharmaceutically acceptable carrier.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration from about 0.1% w/w to about 20% w/w, from about 0.1% w/w to about 10% w/w, from about 0.5% w/w to about 6% w/w, from about 0.75% w/w to about 6% w/w, from about 3% w/w to about 7.5% w/w, from about 3% w/w to about 6% w/w, from about 5% w/w to about 10% w/w, or from about 10% w/w to about 20% w/w, water in a concentration of about 0% w/w to about 4% w/w, and a pharmaceutically acceptable carrier, wherein the non-melanoma skin cancer is selected from a group consisting of skin cancer basal cell carcinoma (BCC), squamous cell skin cancer (SCC), actinic keratosis, Gorlin syndrome, Merkel cell carcinoma, Bowen's disease, classic Kaposi sarcoma, hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 3.5% w/w, about 5% w/w, about 5.5% w/w or about 7% w/w, water in a concentration of about 0% w/w to about 4% w/w, and a pharmaceutically acceptable carrier, wherein the non-melanoma skin cancer is selected from a group consisting of skin cancer basal cell carcinoma (BCC), squamous cell skin cancer (SCC), actinic keratosis, Gorlin syndrome, Merkel cell carcinoma, Bowen's disease, classic Kaposi sarcoma, hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma. Each represents a separate embodiment of this invention.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, water in a concentration of about 0% w/w to about 4% w/w, and a pharmaceutically acceptable carrier. In another embodiment, the erlotinib or a pharmaceutically acceptable salt thereof is in a concentration of from about 0.1% w/w to about 10% w/w, from about 0.5% w/w to about 6% w/w, from about 0.75% w/w to about 5.5% w/w, from about 3% w/w to about 6% w/w, from about 3.5% w/w to about 5% w/w, from about 3.5% to about 7.5% w/w, from about 3.5% w/w to about 5.5% w/w, from about 5% w/w to about 10% w/w, or from about 10% w/w to about 20% w/w. Each represents a separate embodiment of this invention. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of from about 3.5% to about 7.5% w/w, water in a concentration of about 0% w/w to about 4% w/w, and a pharmaceutically acceptable carrier, wherein the non-melanoma skin cancer is selected from a group consisting of skin cancer basal cell carcinoma (BCC), squamous cell skin cancer (SCC), actinic keratosis, Gorlin syndrome, Merkel cell carcinoma, Bowen's disease, classic Kaposi sarcoma, Hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, or sebaceous carcinoma. Each represents a separate embodiment of this invention. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about from about 3.5% to about 7.5% w/w, and a pharmaceutically acceptable carrier, wherein the method does not induce or induces reduced cutaneous side-effects as compared with systemic administration of the same amount of erlotinib or pharmaceutically acceptable salt thereof and wherein said method does not induce cutaneous toxicity or induces reduced cutaneous toxicity of erlotinib or pharmaceutically acceptable salt thereof. In another embodiment, the composition comprises water in a concentration of about 0% w/w to about 4% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of from about 3.5% to about 7.5% w/w, and a pharmaceutically acceptable carrier, and wherein the method does not induce or induces reduced cutaneous side-effects as compared with systemic administration of the same amount of erlotinib or pharmaceutically acceptable salt thereof and wherein said method does not induce cutaneous toxicity or induces reduced cutaneous toxicity of erlotinib or pharmaceutically acceptable salt thereof. In other embodiments, the subject does not suffer from a skin keratinization disorder that requires concomitant treatment of the skin keratinization disorder. In another embodiment, the skin keratinization disorder is genetic skin keratinization disorder. In another embodiment, the composition comprises water in a concentration of about 0% w/w to about 4% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of from about 3.5% to about 7.5% w/w, and a pharmaceutically acceptable carrier, wherein the method does not induce or induces reduced cutaneous side-effects as compared with systemic administration of the same amount of erlotinib or pharmaceutically acceptable salt thereof and wherein said method does not induce cutaneous toxicity or induces reduced cutaneous toxicity of erlotinib or pharmaceutically acceptable salt thereof. In another embodiment, the composition comprises water in a concentration of about 0% w/w to about 4% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of from about 3.5% to about 7.5% w/w, and a pharmaceutically acceptable carrier, as compared with systemic administration of the same amount of erlotinib or pharmaceutically acceptable salt thereof and wherein said method does not induce cutaneous toxicity or induces reduced cutaneous toxicity of erlotinib or pharmaceutically acceptable salt thereof. In other embodiments, the subject does not suffer from a skin keratinization disorder that requires concomitant treatment of the skin keratinization disorder. In another embodiment, the skin keratinization disorder is genetic skin keratinization disorder. In another embodiment, the composition comprises water in a concentration of about 0% w/w to about 4% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of from about 3.5% to about 7.5% w/w, and a pharmaceutically acceptable carrier, wherein the method comprises once daily, twice daily or three times per day topical application of therapeutically effective amounts of the said composition to the skin portion of the subject affected by skin or mucosal disorder is cured, prevented or alleviated or according to doctor's instructions. In another embodiment, the composition comprises water in a concentration of about 0% w/w to about 4% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of from about 3.5% to about 7.5% w/w, and a pharmaceutically acceptable carrier, wherein the method comprises once daily, twice daily or three times per day topical application of therapeutically effective amounts of the said composition to the skin portion of the subject affected by skin or mucosal disorder is cured, prevented or alleviated or according to doctor's instructions. In other embodiments, the subject does not suffer from a skin keratinization disorder that requires concomitant treatment of the skin keratinization disorder. In another embodiment, the skin keratinization disorder is genetic skin keratinization disorder. In another embodiment, the composition comprises water in a concentration of about 0% w/w to about 4% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of from about 3.5% to about 7.5% w/w, and a pharmaceutically acceptable carrier, wherein said topical composition is formulated as a cream, an ointment, a gel, a spray, a shampoo, a skin patch, a mucosal patch, a solution, a lotion or a foam. In another embodiment, the composition comprises water in a concentration of about 0% w/w to about 4% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In another embodiment, said topical composition is formulated as a cream. In another embodiment, said topical composition is formulated as an ointment. In another embodiment, said topical composition is formulated as a gel. In another embodiment, said topical composition is formulated as a spray. In another embodiment, said topical composition is formulated as a shampoo. In another embodiment, said topical composition is formulated as a skin patch. In another embodiment, said topical composition is formulated as a mucosal patch. In another embodiment, said topical composition is formulated as a solution. In another embodiment, said topical composition is formulated as a lotion. In another embodiment, said topical composition is formulated as a foam.

In one embodiment, this invention is directed to a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of from about 3.5% to about 7.5% w/w, and a pharmaceutically acceptable carrier, wherein said topical composition is formulated as a cream, an ointment, a gel, a spray, a shampoo, a skin patch, a mucosal patch, a solution, a lotion or a foam. In another embodiment, said topical composition is formulated as a cream. In another embodiment, said topical composition is formulated as an ointment. In another embodiment, said topical composition is formulated as a gel. In another embodiment, said topical composition is formulated as a spray. In another embodiment, said topical composition is formulated as a shampoo. In another embodiment, said topical composition is formulated as a skin patch. In another embodiment, said topical composition is formulated as a mucosal patch. In another embodiment, said topical composition is formulated as a solution. In another embodiment, said topical composition is formulated as a lotion. In another embodiment, said topical composition is formulated as a foam. In other embodiments, the subject does not suffer from a skin keratinization disorder that requires concomitant treatment of the skin keratinization disorder. In another embodiment, the skin keratinization disorder is genetic skin keratinization disorder. In another embodiment, the composition comprises water in a concentration of about 0% w/w to about 4% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In one embodiment, this invention is directed to a method of treating, preventing or alleviating skin or mucosal disorder, wherein the method comprises topical administration to a subject in need thereof a therapeutically effective amount of a topical composition comprising erlotinib or a pharmaceutically acceptable salt thereof in a concentration of about 0.1% w/w to about 20% w/w, wherein the skin or mucosal disorder is selected from a group consisting of non-melanoma skin cancer, basal cell carcinoma (BCC), squamous cell skin cancer (SCC), actinic keratosis, Gorlin syndrome, Merkel cell carcinoma, Bowen's disease, classic Kaposi sarcoma, hereditary leiomyomatosis (Reed'ds syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, sebaceous carcinoma, keratinization skin disorder, hidradenitis suppurativa, prurigo nodularis or prurigo pigmentosa,. In other embodiments, the subject does not suffer from a skin keratinization disorder that requires concomitant treatment of the skin keratinization disorder. In another embodiment, the skin keratinization disorder is genetic skin keratinization disorder.

In one embodiment, this invention is directed to a method of treating, preventing or alleviating skin or mucosal disorder, wherein the method comprises topical administration to a subject in need thereof a therapeutically effective amount of a topical composition comprising erlotinib or a pharmaceutically acceptable salt thereof in a concentration of about 0.1% w/w to about 20% w/w, wherein said subject does not suffer from a skin keratinization disorder that requires concomitant treatment of the skin keratinization disorder, wherein the skin or mucosal disorder is selected from a group consisting of keratinization skin disorder, pachyonychia congenita, hidradenitis suppurativa, prurigo nodularis or prurigo pigmentosa,. In another embodiment, the keratinization skin disorder is selected from Darier's disease, Hailey-Hailey disease, or palmoplantar keratoderma.

In one embodiment, this invention is directed to a method of treating, preventing or alleviating skin or mucosal disorder, wherein the method comprises topical administration to a subject in need thereof a therapeutically effective amount of a topical composition comprising erlotinib or a pharmaceutically acceptable salt thereof in a concentration of from about 3.5% to about 7.5% w/w, and a pharmaceutically acceptable carrier, wherein the skin or mucosal disorder is selected from a group consisting of basal cell carcinoma (BCC), squamous cell skin cancer (SCC), actinic keratosis, Gorlin syndrome, Merkel cell carcinoma, Bowen's disease, classic Kaposi sarcoma, hereditary leiomyomatosis (Reed'ds syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, sebaceous carcinoma, keratinization skin disorder, hidradenitis suppurativa, prurigo nodularis or prurigo pigmentosa. In another embodiment, the composition comprises water in a concentration of about 0% w/w to about 4% w/w.

In one embodiment, this invention is directed to a method of treating, preventing or alleviating skin or mucosal disorder, wherein the method comprises topical administration to a subject in need thereof a therapeutically effective amount of a topical composition comprising erlotinib or a pharmaceutically acceptable salt thereof in a concentration of from about 3.5% to about 7.5% w/w, and a pharmaceutically acceptable carrier, wherein the skin or mucosal disorder is selected from a group consisting of keratinization skin disorder, pachyonychia congenita, hidradenitis suppurativa, prurigo nodularis or prurigo pigmentosa,. In another embodiment, the keratinization skin disorder is selected from Darier's disease, Hailey-Hailey disease, or palmoplantar keratoderma. In another embodiment, the composition comprises water in a concentration of about 0% w/w to about 4% w/w. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

In another embodiment, the method of treating, preventing or alleviating hyperkeratinization disorder comprises topical administration to a subject in need thereof of a therapeutically effective amount of a topical composition comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from about 3.5% to about 7.5% w/w, from 4% to about 6% w/w, or from about 5% w/w to about 10% w/w erlotinib hydrochloride and from about 10% to about 98% w/w at least one penetration enhancer, or at least one keratolytic agent, or combination thereof. In another embodiment, the composition comprises between 0.1%-40% w/w keratolytic agent.

In another embodiment, the method of treating, preventing or alleviating hyperkeratinization disorder comprises topical administration to a subject in need thereof of a therapeutically effective amount of a topical composition comprising about 0.75% w/w erlotinib hydrochloride, about 70% w/w DMSO, about 25% w/w propylene glycol, about 0.5% w/w 2-phenoxyethanol, about 0.25% w/w methylparaben and about 3% w/w Carbopol 980, wherein the composition is formulated as a gel. In another embodiment, the method of treating, preventing or alleviating hyperkeratinization disorder comprises topical administration to a subject in need thereof of a therapeutically effective amount of a topical composition comprising about 5% w/w erlotinib hydrochloride wherein the composition is formulated as an ointment.

In another embodiment, the method of treating, preventing or alleviating hyperkeratinization disorder comprises topical administration to a subject in need thereof of a therapeutically effective amount of a topical composition comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w, from about 3% w/w to about 5% w/w tapinarof and from about 10% to about 98% w/w at least one penetration enhancer, or at least one keratolytic agent, or combination thereof. In another embodiment, the composition comprises between 0.1%-40% w/w keratolytic agent.

In another embodiment, the method of treating, preventing or alleviating hyperkeratinization disorder comprises topical administration to a subject in need thereof of a therapeutically effective amount of a topical composition comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride , from about 0.01% w/w to about 1% w/w, from about 1% w/w to about 3% w/w or from about 3% w/w to about 5% w/w tofacitinib citrate and from about 10% to about 98% w/w at least one penetration enhancer, or at least one keratolytic agent, or combination thereof. In another embodiment, the composition comprises between 0.1%-40% w/w keratolytic agent.

In another embodiment, the method of treating, preventing or alleviating hyperkeratinization disorder comprises topical administration to a subject in need thereof of a therapeutically effective amount of a topical composition comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3%, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w or from about 3% w/w to about 5% w/w apremilast and from about 10% w/w to about 98% w/w at least one penetration enhancer, or at least one keratolytic agent, or combination thereof. In another embodiment, the composition comprises between 0.1%-40% w/w keratolytic agent.

In other embodiments, the present invention provide a method of treatment, prevention or alleviation of psoriasis, wherein the method comprises administering a therapeutically effective amount of a topical composition comprising from about from about 0.5% w/w to about 3% w/w, from about 3% w/w to about 5% w/w, from about 5% w/w to about 10% w/w, or from about 10% w/w to about 20% w/w erlotinib hydrochloride, from about 0.01% w/w to about 1% w/w, from about 1% w/w to about 3% w/w, from about 3% w/w to about 5% w/w tapinarof and from about 10% to about 98% w/w at least one penetration enhancer, or at least one keratolytic agent, or combination thereof. In another embodiment, the composition comprises between 0.1%-40% w/w keratolytic agent.

In other embodiments, the present invention provide a method of treatment, prevention or alleviation of psoriasis, wherein the method comprises administering a topical composition comprising from about 0.5% w/w to about 3% w/w, from about 3% w/w to about 5% w/w, from about 5% w/w to about 10% w/w, or from about 10% w/w to about 20% w/w erlotinib hydrochloride , from about 0.01% w/w to about 1% w/w, from about 1% w/w to about 3% w/w or from about 3% w/w to about 5% w/w tofacitinib citrate and from about 10% to about 98% w/w at least one penetration enhancer, or at least one keratolytic agent, or combination thereof. In another embodiment, the composition comprises between 0.1%-40% w/w keratolytic agent.

In other embodiments, the present invention provide a method of treatment, prevention or alleviation of psoriasis, wherein the method comprises administering a therapeutically effective amount of a topical composition comprising from about 0.5% w/w to about 3%, from about 3% w/w to about 5% w/w, from about 5% w/w to about 10% w/w, or from about 10% w/w to about 20% w/w erlotinib hydrochloride, from about 0.01% w/w to about 1% w/w roflumilast and from about 10% w/w to about 98% w/w at least one penetration enhancer, or at least one keratolytic agent, or combination thereof. In another embodiment, the composition comprises between 0.1%-40% w/w keratolytic agent.

In some embodiments, the methods as described hereinabove do not induce or induces reduced cutaneous side-effects as compared with the same EGFR inhibitor amount administered in different methods/routes.

In some embodiments, the hyperkeratinization disorder treated, prevented or alleviated within the methods described hereinabove—is selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder and a keratinization mucosal disorder, pachyonychia congenita, hidradenitis suppurativa, Gorlin syndrome, prurigo nodularis, prurigo pigmentosa, nail psoriasis, non-melanoma skin cancer and precancerous skin, mucosal and nail lesions.

According to some embodiments, there is provided a method of treatment, prevention or alleviation of a skin disorder selected from the group consisting of psoriasis, palmoplantar psoriasis, acquired palmoplantar keratosis, eczema, ichtyosis vulgaris, non-melanoma skin cancer, actinic keratosis, pachyonychia congenita, hidradenitis suppurativa, Gorlin syndrome, prurigo nodularis and prurigo pigmentosa by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising at least one additional active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3, a corticosteroid, calcipotriene, tapinarof and combinations thereof in a concentration of from about 0.01% to about 1%, from about 1% to about 3%, from about 3% to about 5% w/w, wherein the at least one EGFR inhibitor and the at least one additional active agent selected from a corticosteroid, calcipotriene, tapinarof and combinations thereof exhibit an additive or synergistic effect.

According to some embodiments, there is provided a method of treatment of a skin or mucosal disorder selected from the group consisting of psoriasis, palmoplantar psoriasis, non-acquired palmoplantar keratosis, eczema, ichthyosis vulgaris, melanoma skin cancer, actinic keratosis, pachyonychia congenita, hidradenitis suppurativa, Gorlin syndrome, prurigo nodularis and prurigo pigmentosa by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising at least one additional active agent selected from a corticosteroid, calcipotriene, tapinarof and combinations thereof in a concentration of from about 0.01% to about 1%, from about 1% to about 3%, from about 3% to about 5% w/w, wherein said at least one EGFR inhibitor and said at least one additional active agent exhibit an additive or synergistic effect.

According to some embodiments, there is provided a method of treatment of psoriasis by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising at least one additional active agent selected from a corticosteroid, calcipotriene, tapinarof and combinations thereof, in a concentration of, from about 0.01% to about 1%, from about 1% to about 3%, from about 3% to about 5% w/w, wherein said at least one EGFR inhibitor and said at least one additional active agent exhibit an additive or synergistic effect.

According to some embodiments, there is provided a method of treatment of a skin disorder selected from the group consisting of psoriasis, palmoplantar psoriasis, acquired palmoplantar keratosis, eczema, ichthyosis vulgaris, non-melanoma skin cancer, actinic keratosis, pachyonychia congenita, hidradenitis suppurativa, Gorlin syndrome, prurigo nodularis and prurigo pigmentosa by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising tapinarof in a concentration of from about 0.01% to about 1%, from about 1% to about 3% or from about 3% to about 5% w/w, wherein said at least one EGFR inhibitor and tapinarof exhibit an additive or synergistic effect.

According to some embodiments, there is provided a method of treatment of palmoplantar psoriasis by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from about 5% to about 10% w/w or from about 10% to about 20% w/w, further comprising tapinarof in a concentration of from about 0.01% to about 1%, from about 1% to about 3% or from about 3% to about 5% w/w, from 4% to about 6% w/w, wherein said at least one EGFR inhibitor and tapinarof exhibit an additive or synergistic effect.

According to some embodiments, there is provided a method any one of treatment of palmoplantar psoriasis by topical administration to a subject in need thereof a therapeutically effective amount of a composition comprising a EGFR inhibitor wherein the EGFR inhibitor is erlotinib in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising tapinarof in a concentration of from about 0.01% to about 1%, from about 1% to about 3% or from about 3% to about 5% w/w, wherein erlotinib and tapinarof exhibit an additive or synergistic effect.

According to some embodiments, there is provided a method of treatment of a skin or mucosal disorder, wherein the skin or mucosal disorder is selected from a keratinization skin disorder and a keratinization mucosal disorder by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising tapinarof in a concentration of from about 0.01% to about 1%, from about 1% to about 3% or from about 3% to about 5% w/w, wherein said at least one EGFR inhibitor and tapinarof exhibit an additive or synergistic effect.

According to some embodiments, there is provided a method of treatment of a keratinization skin disorder or a keratinization mucosal disorder by topical administration to a subject in need thereof a therapeutically effective amount of a composition comprising a EGFR inhibitor wherein the EGFR inhibitor is erlotinib in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w from about 0.01% to about 1%, from about 1% to about 3%, from about 3% to about 5% w/w, from 4% to about 6% w/w, from about 5% to about 10% w/w or from about 10% to about 20% w/w, further comprising tapinarof in a concentration of from about 0.01% to about 1%, from about 1% to about 3% or from about 3% to about 5% w/w, wherein erlotinib and tapinarof exhibit an additive or synergistic effect.

In some embodiments, there is provided a method of treatment comprising daily, twice daily or three times per day application of therapeutically effective amounts of the composition or the two or three separate compositions to the skin portion of the subject affected by the said skin or mucosal disorder until the skin or mucosal disorder is cured, prevented or alleviated or according to doctor's instructions. In one embodiment, the method comprises daily, twice daily or three times per day topical application of therapeutically effective amounts of the topical composition or the two or three separate topical compositions to the skin portion of the subject affected by the said skin or mucosal disorder until the skin or mucosal disorder is cured, prevented or alleviated or according to doctor's instructions.

In one embodiment, the administration of the compositions within the above methods is topical or injectable administration (subcutaneous, intramuscular, intravenous, intraperitoneal, intracardiac, intraarticular, intracavernous or intralesional administration, each represents a separate embodiment) intramuscular, intravenous, intraperitoneal, intracardiac, intraarticular, intracavernous or intralesional administration, each represents a separate embodiment). In another embodiment, the administration is topical or intralesional. In another embodiment, intralesional administration is done by regular injections or subcutaneous injections with microneedles. In another embodiment, the compositions are topical or injectable. Each possibility represents a separate embodiment of this invention. In some other embodiments, the EGFR inhibitor in any of the methods and compositions of this invention is erlotinib or salt thereof. In another embodiment the EGFR inhibitor is Erlotinib HCl. In other embodiments the erlotinib salts include a salt with an inorganic or organic acid, such as, hydrochloric, hydrobromic, sulphuric, phosphoric, methanesulfonic, benzenesulfonic, trifluoroacetic, citric, lactic, maleic acid, tosylic or oxalic acid.

As EGFR inhibitors in general and erlotinib in particular are poorly soluble, the compositions of this invention need to comprise a high EGFR inhibitor concentration of up to 20% w/w. The compositions are in the form of partly solubilized suspensions and may comprise organic solvents and solubility enhancers as well as other ingredients (e.g. penetration enhancer) and active agents which are all described hereinabove. In one embodiment, the at least one EGFR inhibitor is partly or entirely solubilized.

Unexpectedly, it was found that within the compositions of this invention, EGFR inhibitors were at least partially solubilized and used in solution/suspension form, thus the inhibitors could be used in topical or injectable compositions. Further, it was found that the compositions of this invention, when administered topically or intralesional injected within the methods described hereinabove—do not induce or induces reduced cutaneous side-effects as compared with the same EGFR inhibitor amount administered in different methods/routes.

In some embodiments, the composition of this invention, when administered topically within the methods described hereinabove do not induce side effects. In some embodiments, the composition of this invention, when administered topically within the methods described hereinabove do not induce adverse events. In some embodiments, the composition of this invention, when administered topically within the methods described hereinabove, induces only “mild” adverse events.

In some embodiments, the composition comprising erlotinib, when administered topically within the methods described hereinabove, induces only “mild” adverse events.

The term “Mild” adverse events refer to adverse events that are usually transient, requiring no special treatment, and do not interfere with subject's daily activities.

In some embodiments, when topically applying the composition of this invention there is no systemic absorbance of erlotinib and its metabolite. In another embodiment, no systemic absorbance of erlotinib and its metabolite refers to below the limit detection of erlotinib in the plasma. In another embodiment, the limit detection is 97.73 ng/mL (for erlotinib) and 9.783 ng/mL (for erlotinib metabolite), for topically administering 5% and 3.5% of erlotinib ointment.

In some embodiments, a method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w, wherein said subject does not suffer from a skin keratinization disorder that requires concomitant treatment of the skin keratinization disorder, wherein the method comprises once daily, twice daily or three times per day topical application of therapeutically effective amounts of the said composition to the skin portion of the subject affected by skin or mucosal disorder is cured, prevented or alleviated or according to doctor's instructions. In another embodiment, the topical composition has a systemic absorption of less than 100 ng/mL of erlotinib or less than 10 ng/mL of erlotinib metabolite in the plasma. In another embodiment, the topical composition has a systemic absorption of less than 1% or less than 0.5% by weight of erlotinib or pharmaceutically acceptable salt thereof.

### Definitions

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

As used herein, the term “non-melanoma skin cancer” refers to non-melanoma skin cancer disorders such as basal layer (BCC), squamous layer (SCC), Merkel cell carcinoma, Kaposi sarcoma skin lymphoma and to diseases related to non-melanoma skin cancer diseases that for an internal factor such as genetics, hereditary or even external factor such as the sun, repeated trauma they can be transformed into BCC or SCC.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”. As used herein, numerical ranges preceded by the term “about” should not be considered to be limited to the recited range. Rather, numerical ranges preceded by the term “about” should be understood to include a range accepted by those skilled in the art for any given element in microcapsules or formulations according to this invention.

The term “about” as used herein means within an acceptable error range for a particular value as determined by one of ordinary skill in the art, which will depend in part on how the value is measured or determined, i.e., the limitations of the measurement system. For example, “about” can mean a range of up to 10%, more preferably up to 5%, and still more preferably up to 1% of a given value. Where particular values are described in the application and claims, unless otherwise stated, the meaning of the term “about” is within an acceptable error range for the particular value.

The terms “comprise”, “comprising”, “includes”, “including”, “having” and their conjugates mean “including but not limited to”.

The term “consisting of” means “including and limited to”.

The term “consisting essentially of” means that the composition, method or microcapsules may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

### Example 1

### Preparation and Stability of a 0.75% Topical Erlotinib HCl Gel Composition

0.75% erlotinib; 70% DMSO;

### Composition

| Ingredient | % in formulation |
|---|---|
| Erlotinib hydrochloride | 0.75 |
| DMSO | 70 |
| Propylene glycol | 25.50 |
| 2-phenoxyethanol | 0.5 |
| Methylparaben | 0.25 |
| Carbopol 980 | 3 |

### Procedure

Erlotinib hydrochloride was dissolved in DMSO at 40° C.
Methylparaben was added under stirring
Carbopol was added under stirring
2-phenoxyethanol was dissolved in propylene glycol and added
The formulation was stirred and homogenized to obtain a homogeneous gel.

### Stability Results

| | Time | 1 month | 2 months | 3 months |
|---|---|---|---|---|
| | zero | at 40 C. | at 40 C. | at 40 C. |
| Erlotinib assay | 0.70% | 0.71% | 0.71% | 0.73% |

### Example 2

### Preparation and Stability of a 0.5% Topical Erlotinib HCl Gel Composition

0.5% erlotinib; 70% DMSO;

### Composition

| Ingredient | % in formulation |
|---|---|
| Erlotinib hydrochloride | 0.5 |
| DMSO | 70 |
| Propylene glycol | 25.75 |
| 2-phenoxyethanol | 0.5 |
| Methylparaben | 0.25 |
| Carbopol 980 | 3 |

### Procedure

Erlotinib hydrochloride was dissolved in DMSO at 40° C.
Methylparaben was added under stirring
Carbopol was added under stirring
2-phenoxyethanol was dissolved in propylene glycol and added
The formulation was stirred and homogenized to obtain a homogeneous gel.

### Stability Results

| | Time zero | 2 months at 40 C. | 3 months at 40 C. |
|---|---|---|---|
| Erlotinib assay | 0.46% | 0.47% | 0.46% |

### Example 3

### Preparation and Stability of a 0.5% Topical Erlotinib HCl Gel Composition

0.5% erlotinib; 45.5% DMSO

### Composition

| Ingredient | % in formulation |
|---|---|
| Erlotinib hydrochloride | 0.5 |
| DMSO | 45.5 |
| Propylene glycol | 50.25 |
| 2-phenoxyethanol | 0.5 |
| Methylparaben | 0.25 |
| Carbopol 980 | 3 |

### Procedure

Erlotinib hydrochloride was dissolved in DMSO at 40° C.
Methylparaben was added under stirring
Carbopol was added under stirring
2-phenoxyethanol was dissolved in propylene glycol and added
The formulation was stirred and homogenized to obtain a homogeneous gel.

### Stability Results

| | Time zero | 2 months at 40 C. | 3 months at 40 C. |
|---|---|---|---|
| Erlotinib assay | 0.47% | 0.47% | 0.46% |

### Example 4

### Preparation and Stability of a 0.5% Topical Erlotinib HCl Gel Composition

0.5% erlotinib; 50% EtOH 70%

### Composition

| Ingredient | % in formulation |
|---|---|
| Erlotinib hydrochloride | 0.5 |
| EtOH 70% | 50 |
| Propylene glycol | 46.25 |
| 2-phenoxyethanol | 0.5 |
| Methylparaben | 0.25 |
| Carbopol 980 | 2.5 |

### Procedure

Erlotinib hydrochloride was dissolved in EtOH at 40° C.
Methylparaben was added under stirring
Carbopol was added under stirring
2-phenoxyethanol was dissolved in propylene glycol and added
The formulation was stirred and homogenized to obtain a homogeneous gel.

### Stability Results

| | Time zero | 2 weeks at 40 C. |
|---|---|---|
| Erlotinib assay | 0.47% | 0.48% |

### Example 5

### Preparation and Stability of a 1.25% Topical Erlotinib HCl Gel Composition

1.25% erlotinib; 95% DMSO;

### Composition

| Ingredient | % in formulation |
|---|---|
| Erlotinib hydrochloride | 1.25 |
| DMSO | 95 |
| 2-phenoxyethanol | 0.5 |
| Methylparaben | 0.25 |
| Carbopol 980 | 3 |

### Procedure

Erlotinib hydrochloride was dissolved in DMSO at 40° C.
Methylparaben was added under stirring.
Carbopol was added under stirring.
2-phenoxyethanol was dissolved in propylene glycol and added
The formulation was stirred and homogenized to obtain a homogeneous gel.

### Example 6

### Preparation and Stability of a 1% Topical Erlotinib HCl Gel Composition

1% erlotinib; 49% PEG-400; 30% PEG-3350

### Composition

| Ingredient | % in formulation |
|---|---|
| Erlotinib hydrochloride | 1 |
| Propylene glycol | 20 |
| PEG-400 | 49 |
| PEG-3350 | 30 |

### Procedure

Propylene glycol, PEG-400 and PEG-3350 were stirred at 70% to obtain a homogeneous liquid
Erlotinib hydrochloride was added under stirring
Carbopol was added under stirring and homogenization
2-phenoxyethanol was dissolved in propylene glycol and added
The formulation was cooled to room temperature.

### Example 7

### Preparation and Stability of a 1% Erlotinib HCl+1% Tapinarof Topical Gel Composition

| Ingredient | % in formulation |
|---|---|
| Erlotinib hydrochloride | 1 |
| Tapinarof | 1 |
| DMSO | 70 |
| Propylene glycol | 24.25 |
| 2-phenoxyethanol | 0.5 |
| Methylparaben | 0.25 |
| Carbopol 980 | 3 |

### Procedure

Erlotinib hydrochloride is dissolved in DMSO at 40° C.
Tapinarof is added under stirring
Methylparaben is added under stirring
Carbopol is added under stirring
2-phenoxyethanol is dissolved in propylene glycol and added
The formulation is stirred and homogenized to obtain a homogeneous gel.

### Example 8

### Preparation of a 1% Erlotinib HCl+0.5% Tofacitinib Citrate Topical Gel Composition

| Ingredient | % in formulation |
|---|---|
| Erlotinib hydrochloride | 1 |
| tofacitinib citrate | 0.5 |
| DMSO | 70 |
| Propylene glycol | 24.75 |
| 2-phenoxyethanol | 0.5 |
| Methylparaben | 0.25 |
| Carbopol 980 | 3 |

### Procedure

Erlotinib hydrochloride is dissolved in DMSO at 40° C.
Tofacitinib citrate is added under stirring
Methylparaben is added under stirring
Carbopol is added under stirring
2-phenoxyethanol is dissolved in propylene glycol and added
The formulation is stirred and homogenized to obtain a homogeneous gel.

### Example 9

### Preparation of a 1% Erlotinib HCl+0.5% Apremilast Topical Gel Composition

| Ingredient | % in formulation |
|---|---|
| Erlotinib hydrochloride | 1 |
| Apremilast | 0.5 |
| DMSO | 70 |
| Propylene glycol | 24.75 |
| 2-phenoxyethanol | 0.5 |
| Methylparaben | 0.25 |
| Carbopol 980 | 3 |

### Procedure

Erlotinib hydrochloride is dissolved in DMSO at 40° C.
Apremilast is added under stifling
Methylparaben is added under stirring
Carbopol is added under stirring
2-phenoxyethanol is dissolved in propylene glycol and added
The formulation is stirred and homogenized to obtain a homogeneous gel.

### Example 10

### Preparation of a 5% Erlotinib HCl Ointment

| Ingredient | % in formulation |
|---|---|
| Erlotinib hydrochloride | 5 |
| PEG 400 | 62.85 |
| PEG 3350 | 25 |
| Glycerin | 4.1 |
| 2-phenoxyethanol | 0.5 |
| Methylparaben | 0.25 |
| NaOH 20% solution | 2.3 |

### Procedure

Erlotinib hydrochloride was added to PEG-400 at 50° C.
Sodium hydroxide solution was added until full dissolution is obtained.
Methylparaben, phenoxyethanol, glycerin were added under stirring.
PEG-3350 was added.
The formulation was stirred and cooled to room temperature during mixing to obtain a homogeneous ointment.

### Example 11

### Efficacy Study of Erlotinib 5% for the Treatment of Psoriasis

The objective of this study was to determine the efficacy of Erlotinib 5% as provided herein compared to the vehicle and Dexamethasone as shown at the Table below. The evaluation was based on histology analysis following application of the respective test formulations.

| | Active | Dosage |
|---|---|---|
| Group | ingredient | form |
| 1 | Vehicle | Ointment |
| 2 | Dexamethasone | 5 Solution |
| | 2 mg | |
| 3 | Erlotinib | Ointment |
| | 5% | |

The efficacy study has been conducted on female mice. The total duration of the experiment was 8 weeks, upon receiving of the human skin. Normal skin from healthy volunteers were obtained for grafting. Healthy human abdominal skin pieces with a width of 0.4 mm and surface area of 1.5×1.5 cm were provided. The skin sample was preserved in isotonic saline solution and transplanted within 6-12 hours after skin donation. In addition, blood samples were collected from psoriatic patients having classic plaque psoriasis and not undergoing any treatment. 20 mL blood samples were taken from both males and females psoriatic patients (from psoriatic patients having classic plaque psoriasis and not undergoing any treatment), and peripheral blood mononuclear cells (PBMC) were separated immediately after blood withdrawal.

Mice: Beige-severe combined immunodeficient mice C.B-17/IcrHsdscid-bg (beige-SCID) mice (weight ˜25 g) were included in this study. Mice were monitored twice a week for vital signs such as weight, food intake, coat, eyes, anal genital areas, discharge/soiling, behavior and criteria related to skin transplantation. Normal healthy human donor skin were transplanted onto the beige-SCID mice as previously described (See Keren A. et al., Novel nanosome delivery system combined with siRNA targeting the antimicrobial gene DFB4: a new approach for psoriasis management?ExpDermatol. 2014; 23:464-465; Bracke S. et al.,Targeted silencing of DEFB4 in a bioengineered skin-humanized mouse model for psoriasis: development of siRNA SECosome-based novel therapies. ExpDermatol. 2014 ;23:199-201; Zaretsky M. et al., Directed evolution of a soluble human IL-17A receptor for the inhibition of psoriasis plaque formation in a mouse model. Chem Biol. 2013 21; 20:202-2; Gilhar A. et al., The beneficial effect of blocking Kv1.3 in the psoriasiform SCID mouse model. J Invest Dermatol. 2011;131:118 124; Keren A. et al., Innate lymphoid cells 3 induce psoriasis in xenotransplanted healthy human skin. J Allergy Clin Immunol. 2018;142:305-308.e6.).

PBMC's from the psoriatic patient's blood were isolated and cultured in the presence of a high dose of IL-2; Prospec, 100 U/mL of media-RPMI 1640, 10% human AB serum (Sigma, St.Louis, Mo.), 1% glutamine, 1% antibiotics (media components; Biologicallndustries, Kibbutz Beit Haemeck, Israel), as previously described (Gilhar A. et al., The beneficial effect of blocking Kv1.3 in the psoriasiform SCID mouse model. J Invest Dermatol. 2011;131:118-124; Nousbeck J. et al., IGFBP7 as a potential therapeutic target in Psoriasis. J Invest Dermatol. 2011;131:1767-1770; Schafer P H. et al., Apremilast, a cAMP phosphodiesterase-4 inhibitor, demonstrates anti-inflammatory activity in vitro and in a model of psoriasis. British Journal of Pharmacology 2010; 159:842-855).

Four weeks following the engraftment, each mouse was injected (intradermally) with 1×10⁷ IL-2 enriched allogeneic PBMC's from psoriatic patients (1×10⁷ cells injected/mouse). Cells from different psoriasis patients were equally distributed between treatment groups. Each patient was represented in each treatment group.

Fourteen days following cells injections, the mice were divided into treatment groups (Table, see below) and treated according to the timeline indicated below (see Scheme 1). Each transplant was equally topically treated (50 μg) by using a sterile spatula.

Dexamethasone (D2915, Sigma): was freshly prepared for each administration by dilution in saline to final concentration (50 mg/ml). Dexamethasone serves as a positive control and was administered topically twice daily for 14 days following cells injections (at Day 42 following human skin transplantation) until the end of the study (Day 56).

The vehicle was administered topically (50 μg) once daily for 14 days as well.

On Day 56, the entire skin graft was excised and placed in 10% formaldehyde in saline overnight. Then, the specimens were placed in 70% ethanol and embedded and stained according to the standard Hematoxylin/Eosin protocol.

Skin graft histological assessment was performed (according to the psoriatic criteria including epidermal thickness) using light microscopy, and two observers blindly performed the evaluations. Epidermal thickness was determined using an ocular micrometer at a minimum of 50 points along the epidermis selected to represent points of maximal and minimal thickness. Thickness of the suprapapillary plate was measured similarly at 50 points for each sample. Three slices were examined for each sample.

### Study Results

### Histological Evaluation of Psoriasis-Induced Xenotransplants Study

| | | | Fre- | Psoriatic | Partial | Complete |
|---|---|---|---|---|---|---|
| Group | Compound | Route | quency | Features | Recovery | Recovery |
| 1 | Vehicle | Topical | Once | 9/10 | 1/10 | 0/10 |
| | ointment | | daily | (90%) | (10%) | (0%) |
| 2 | Dexa- | Topical | Twice | 0/10 | 1/10 | 9/10 |
| | methasone | | a day | (0%) | (10%) | (90%) |
| | 2 mg | | | | | |
| 3 | Erlotinib | Topical | Once | 4/10 | 1/10 | 5/10 |
| | ointment | | daily | (40%) | (10%) | (50%) |
| | 5% | | | | | |

### Example 12

### Epidermal Proliferation of Transplants Treated with Erlotinib 5% Ointment

### Immunohistochemical Staining

From the study of Example 11, the human skin xenotransplants were harvested and fixed in 10% saline-buffered formalin overnight followed by 70% ethanol. Sections were stained and visualized with immunohistochemistry.

Antigen retrieval was performed on the slides for 20 minutes in a microwave followed by cooling at room temperature for 25 minutes. Specimens were blocked for 30 minutes to prevent nonspecific binding and incubated with the primary antibody (Ab) overnight. The following primary antibodies were used: mouse anti human Ki67. Samples were then washed and incubated for 30 minutes with a biotinylated secondary antibody (Jackson ImmunoResearch, West Grove, Pa.). Finally, samples were washed and incubated with streptavidin-horseradish peroxidase (Jackson ImmunoResearch, West Grove, Pa.). The proteins were revealed by treating the sections with 3-amino-9-ethylcarbaz,ole. Samples were examined using light microscopy.

Slides were evaluated by experienced blinded observers. The fields of immunostained sections were counted in three areas and presented randomly to the observer(s).

### Results

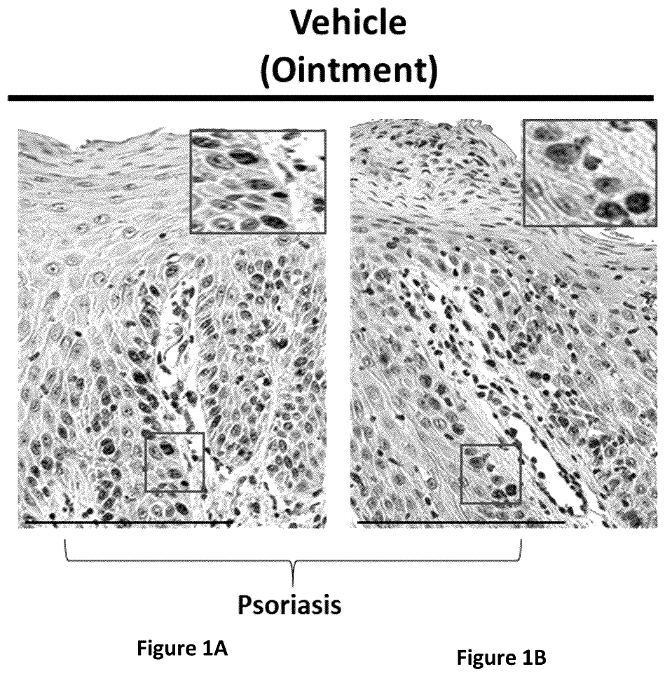

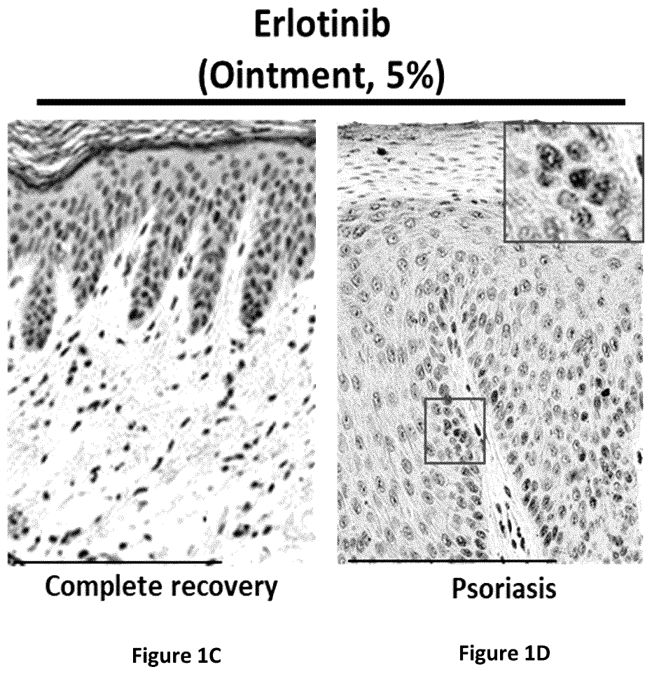

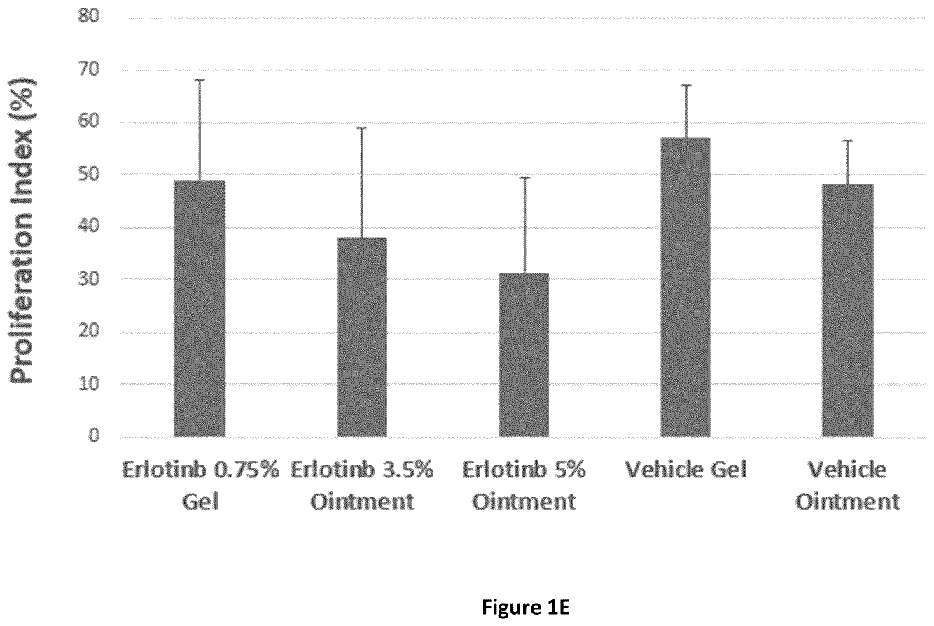

A significant decrease in proliferation index (Ki67) in Erlotinib-treated eno ansplants compared to vehicle-treated xenotransplants as shown in FIGS. 1A-1E.

The results of the proliferation index (%) presented in FIG. 1E is summarized in the following Table:

| | Erlotinib | Erlotinib | Erlotinib | | |
|---|---|---|---|---|---|
| | 0.75% | 3.5% | 5% | Vehicle | Vehicle |
| | Gel | Ointment | Ointment | Gel | Ointment |
| Proliferation | 49 | 38 | 31.4 | 57 | 48.2 |
| Index (%) | | | | | |
| STDEV | 19 | 21 | 18 | 10 | 8.17 |

As can be seen a significant decrease proliferation index (Ki-67) in Erlotinib-treated xenotransplants were obtained with 3.5% w/w and 5% w/w erlotinib.

### Example 13

### Maximal Use Systemic Exposure (MUSE) Study to Evaluate the Pharmacokinetics (PK), Safety and Tolerability of Erlotinib 5% Ointment and Erlotinib 3.5% Ointment in Healthy Volunteer Subjects

### Study Design

The study was designed to determine Erlotinib drug and its active metabolite, OSI-420 (Desmethyl Erlotinib) having the following structure:

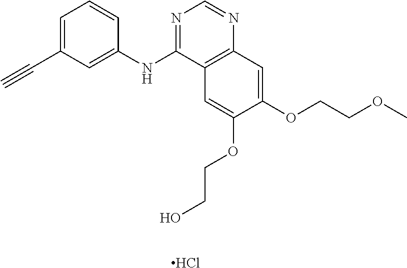 [SMILES: C#Cc1cccc(Nc2ncnc3cc(OCCOC)c(OCCO)cc23)c1 ; Cl]

concentrations in plasma following single and repeated doses of ointment composition of Erlotinib 5% and 3.5% w/w, when applied topically once daily for 28 days under maximum use conditions in Healthy Volunteer Subjects between the ages of 18-65.

The study was also designed to evaluate the safety and tolerability profile of Erlotinib in both doses.

12 enrolled subjects were assigned to the Erlotinib drug in one of two doses, 3.5% and 5%, in a 1:1 ratio.

The assigned study treatment was topically applied to about 15% body surface area (BSA) once daily for 28 days.

Pharmacokinetics blood samples were taken along 28 days of treatment and 2 days of follow-up.

No systemic absorbance was defined as where the mean level of erlotinib and its known metabolite (OSI-420), in each time point, and where the Cmax is less than the method quantitation level of 97.73 ng/mL and 9.783 ng/mL respectively.

The determination of Erlotinib and its metabolite OSI-420 in Human plasma was performed by HPLC MS/MS.

### Study Results

The study results demonstrated that the systemic absorbance of erlotinib and its metabolite was below the lowest quantitation level for both doses (97.73 ng/mL and 9.783 ng/mL respectively). No PK analysis was performed since no absorbance was demonstrated.

Study drug-related adverse events were mainly designated as mild, and all were resolved during the study duration.

### Example 14

### Preparation of a 5.46% Erlotinib HCl Ointment

| Ingredient | % |
|---|---|
| Erlotinib hydrochloride | 5.46 |
| PEG 400 | 66.10 |
| PEG 3350 | 25.00 |
| 2-Phenoxyethanol | 0.50 |
| Methylparaben* | 0.25 |
| NaOH 20% solution | 2.69 |

### Procedure

Erlotinib hydrochloride was added to PEG-400 at 50° C.
Sodium hydroxide solution was added until full dissolution is obtained.
Methylparaben and phenoxyethanol, were added under stirring.
PEG-3350 was added.
The formulation was stirred and cooled to room temperature during mixing to obtain a homogeneous ointment.

What is claimed is:

1. A method of treating, preventing or alleviating non-melanoma skin cancer, wherein the method comprises topical administration to a subject in need thereof a therapeutically effective amount of a topical composition comprising erlotinib or a pharmaceutically acceptable salt thereof in a concentration of about 0.1% w/w to about 20% w/w.

2. The method of claim 1, wherein the non-melanoma skin cancer is selected from a group consisting of skin cancer basal cell carcinoma (BCC), squamous cell skin cancer (SCC), actinic keratosis, Gorlin syndrome, Merkel cell carcinoma, Bowen's disease, classic Kaposi sarcoma, Hereditary leiomyomatosis (Reed's syndrome), Ankyloblepharon-ectodermal defects-cleft lip/palate (AEC) syndrome, dermatofibrosarcoma protuberans, granular cell tumors on skin, disseminated superficial actinic porokeratosis (DSAP), Sezary syndrome, keratocystic odontogenic tumor, epidermolysis Bullosa-associated squamous cell carcinoma, angiosarcoma of the skin, cutaneous B-cell lymphoma, cutaneous T-cell lymphoma, and sebaceous carcinoma.

3. The method according to claim 1, wherein said subject does not suffer from a skin keratinization disorder that requires concomitant treatment of the skin keratinization disorder.

4. The method according to claim 3, wherein the skin keratinization disorder is genetic skin keratinization disorder.

5. The method according to claim 1, wherein the method does not induce or induces reduced cutaneous side-effects as compared with systemic administration of the same amount of erlotinib or pharmaceutically acceptable salt thereof and wherein said method does not induce cutaneous toxicity or induces reduced cutaneous toxicity of erlotinib or pharmaceutically acceptable salt thereof.

6. The method of claim 1, wherein the administration of the topical composition has a systemic absorption of less than 100 ng/mL of erlotinib or less than 10 ng/mL of erlotinib metabolite.

7. The method of claim 1, wherein the administration of the topical composition has a systemic absorption of less than 1% or less than 0.5% by weight of erlotinib or pharmaceutically acceptable salt thereof.

8. The method according to claim 1, wherein the method comprises once daily, twice daily or three times per day topical application of therapeutically effective amounts of the said composition to the skin portion of the subject affected by non-melanoma skin cancer is cured, prevented or alleviated or according to doctor's instructions.

9. The method of claim 1, wherein said topical composition is formulated as a cream, an ointment, a gel, a spray, a shampoo, a skin patch, a mucosal patch, a solution, a lotion or a foam.

10. The method of claim 1, wherein said topical composition comprises water in a concentration of about 0% w/w to about 4% w/w.

11. A topical composition comprising erlotinib or a pharmaceutically acceptable salt thereof in a concentration of about 3.5%-7.5% w/w, and a pharmaceutically acceptable carrier, wherein administration of said composition does not induce or induces reduced cutaneous side-effects as compared with systemic administration of the same amount of erlotinib or pharmaceutically acceptable salt thereof and wherein said administration does not induce cutaneous toxicity or induces reduced cutaneous toxicity of erlotinib or pharmaceutically acceptable salt thereof.

12. The topical composition of claim 11, wherein the topical composition has a systemic absorption of less than 100 ng/mL of erlotinib or less than 10 ng/mL of erlotinib metabolite.

13. The topical composition of claim 11, wherein the topical composition has a systemic absorption of less than 1% or less than 0.5% by weight of erlotinib or pharmaceutically acceptable salt thereof.

14. The topical composition of claim 11, wherein the composition comprises water in a concentration of about 0% w/w to about 4% w/w.

15. The topical composition of claim 11, further comprising one or more gelling agent in an amount of about 0.1% w/w to about 6% w/w of the topical composition, and wherein the composition is anhydrous.

16. The topical composition of claim 11, wherein the topical composition comprises erlotinib or a pharmaceutically acceptable salt thereof in a concentration of about 3.5% w/w, about 5% w/w, about 5.5% w/w or about 7% w/w.

17. The topical composition of claim 11, wherein the topical composition is formulated as a cream, an ointment, a gel, a spray, a shampoo, a skin patch, a mucosal patch, a solution, a lotion or a foam.

18. The topical composition of claim 17, wherein the erlotinib is formulated as an ointment or a gel.

19. A method of treating, preventing or alleviating non-melanoma skin cancer, wherein the method comprises topical administration to a subject in need thereof a therapeutically effective amount of a topical composition according to claim 11.

20. The method of claim 19, wherein the non-melanoma skin cancer is skin cancer basal cell carcinoma (BCC), squamous cell skin cancer (SCC), actinic keratosis, Gorlin syndrome, Merkel cell carcinoma, Bowen's disease, Classic Kaposi sarcoma, Hereditary leiomyomatosis (Reed'ds syndrome), Ankyloblepharon-ectodermal defects-cleft lip/palate (AEC) syndrome, Dermatofibrosarcoma protuberans, Granular cell tumors on skin, Disseminated Superficial Actinic Porokeratosis (DSAP), Sezary syndrome, Keratocystic Odontogenic Tumor, and Epidermolysis Bullosa-Associated Squamous Cell Carcinoma Angiosarcoma of the skin, Cutaneous B-cell lymphoma, Cutaneous T-cell lymphoma, or Sebaceous carcinoma.

21. The method according to claim 19, wherein said subject does not suffer from a skin keratinization disorder that requires concomitant treatment of the skin keratinization disorder.

22. The method according to claim 19, wherein the skin keratinization disorder is genetic skin keratinization disorder.

23. The method according to claim 19, wherein the method does not induce or induces reduced cutaneous side-effects as compared with systemic administration of the same amount of erlotinib or pharmaceutically acceptable salt thereof and wherein said method does not induce cutaneous toxicity or induces reduced cutaneous toxicity of erlotinib or pharmaceutically acceptable salt thereof.

24. The method according to claim 19, wherein the administration of the topical composition has a systemic absorption of less than 100 ng/mL of erlotinib or less than 10 ng/mL of erlotinib metabolite.

25. The method according to claim 19, wherein the administration of the topical composition has a systemic absorption of less than 1% or less than 0.5% by weight of erlotinib or pharmaceutically acceptable salt.

26. The method according to claim 19, wherein the administration of the topical composition reduces the proliferation index (Ki-67).

27. The method according to claim 19, wherein the method comprises once daily, twice daily or three times per day topical application of therapeutically effective amounts of the said composition to the skin portion of the subject affected by non-melanoma skin cancer is cured, prevented or alleviated or according to doctor's instructions.

28. The method of claim 19, wherein said topical composition is formulated as a cream, an ointment, a gel, a spray, a shampoo, a skin patch, a mucosal patch, a solution, a lotion or a foam.
